(19) United States
(12) Patent Application Publication
(10) Pub. No.: US 2021/0187071 A1
(43) Pub. Date: Jun. 24, 2021

(54) COMPOSITIONS COMPRISING MDA-7/IL-24 PROTEIN AND METHODS OF USE

(71) Applicant: VIRGINIA COMMONWEALTH UNIVERSITY, Richmond, VA (US)

(72) Inventors: Paul B. FISHER, Richmond, VA (US); Anjan K. PRADHAN, Richmond, VA (US); Swadesh K. DAS, Richmond, VA (US); Luni EMDAD, Richmond, VA (US)

(21) Appl. No.: 16/754,799

(22) PCT Filed: Oct. 26, 2018
(86) PCT No.: PCT/US2018/057708

§ 371 Date: Apr. 9, 2020

Related U.S. Application Data
(60) Provisional application No. 62/577,932, filed on Oct. 27, 2017.
(60) Provisional application No. 62/687,905, filed on Jun. 21, 2018.

(51) Int. Cl. A61K 38/20; A61K 31/122; A61K 31/5377; A61P 35/04

(52) U.S. Cl. CPC A61K 38/20; A61P 35/04; A61K 31/5377; A61K 31/122

(57) ABSTRACT

In various aspects, the present disclosure provides methods of preventing metastasis to bone in a subject with cancer, as well as compositions and kits for use in the same. In various aspects, the present disclosure provides methods of treating bone metastasis in a subject with cancer, as well as compositions and kits for use in the same. In embodiments, compositions comprise an MDA-7/IL-24 protein.

### CROSS-REFERENCES TO RELATED APPLICATIONS

This application claims the benefit of U.S. Provisional Application No. 62/577,932, filed Oct. 27, 2017, and U.S. Provisional Application No. 62/687,905, filed Jun. 21, 2018, which are hereby incorporated by reference in their entireties and for all purposes.

### SEQUENCE LISTING

The Sequence Listing written in file 053151-503001WO_Sequence_Listing_ST25.txt, created on Oct. 25, 2018, 5,694 bytes, machine format IBM-PC, MS-Windows operating system, is hereby incorporated by reference.

### BACKGROUND

For many malignancies, mortality derives from widespread metastases, such as metastasis to bone. Bone metastases are also associated with severe morbidity, pain, and functional impairment. No current single or combinatorial therapeutic approach has been effective in decreasing morbidity or engendering a cure for metastases to bone.

Prostate cancer (PC) is one of the most common cancers affecting men worldwide with a strong propensity for bone metastases, which are refractory to conventional therapeutic approaches (1). Currently, advanced PC is incurable and results in significant disease morbidity and mortality (2). Bone metastasis begins with the dissemination of tumor cells towards bone, adherence to bone marrow cells, penetration/invasion into bone marrow to the mineralized matrix, and growth of micro-metastatic lesions (3). Colonization of cancer cells in bone is regulated by a variety of factors that determine the extent to which cancer cells can engage and communicate with the bone marrow, particularly with osteoblast and osteoclast cells, which are the two major components of tumor bone modeling (4). Understanding the molecular factors influencing this multistep process and the associated signaling pathways remain critical to designing effective therapeutics to inhibit and treat bone metastasis.

### BRIEF SUMMARY

In view of the foregoing, there is a need for improved methods for preventing and treating bone metastasis of cancer. The present disclosure provides methods and compositions that address this need, and provide additional benefits as well.

In some aspects, the present disclosure provides a method of preventing metastasis to bone in a subject with cancer. In some aspects, the present disclosure provides a method of treating bone metastasis in a subject with cancer. In embodiments, the method comprises administering an effective amount of a composition comprising an MDA-7/IL-24 protein to the subject. In embodiments, the MDA-7/IL-24 protein is a purified protein. In embodiments, the MDA-7/IL-24 protein is a mature protein. In embodiments, the administering comprises administering to a bone of said subject. In embodiments, the cancer is prostate cancer. In embodiments, the prostate cancer comprises cancer cells having an increased expression of one or more of Mcl-1, RANKL, Bcl-2, Bcl-xL, and Akt, relative to normal prostate cells. In embodiments, the composition further comprises an Mcl-1 inhibitor, such as BI-97D6. In embodiments, the composition further comprises a phosphoinositide 3-kinase (PI3K) inhibitor, such as LY294002. In embodiments, the effective amount is an amount that is substantially non-toxic to primary bone marrow cells or normal primary human prostate epithelial cells. In embodiments, the effective amount is an amount that inhibits osteoclast differentiation. In embodiments, the MDA-7/IL-24 protein comprises an amino acid sequence that is at least 90% identical to SEQ ID NO: 3 (e.g., at least 95% identical). In embodiments, the MDA-7/IL-24 protein is capable of activating an IL-20/IL-22 receptor complex of a cancer cell of the subject. In embodiments, the MDA-7/IL-24 protein comprises an amino acid sequence of SEQ ID NO: 3.

In some aspects, the present disclosure provides a composition. In embodiments, the composition comprises an MDA-7/IL-24 protein and one or both of an Mcl-1 inhibitor and a PI3K inhibitor. In embodiments, the MDA-7/IL-24 protein is a purified protein. In embodiments, the MDA-7/IL-24 protein is a mature protein. In embodiments, the composition comprises an Mcl-1 inhibitor (e.g., BI-97D6). In embodiments, the composition comprises a PI3K inhibitor (e.g., LY294002). In embodiments, the MDA-7/IL-24 protein comprises an amino acid sequence that is at least 90% identical to SEQ ID NO: 3 (e.g., at least 95% identical). In embodiments, the MDA-7/IL-24 protein is capable of activating an IL-20/IL-22 receptor complex of a cancer cell. In embodiments, the MDA-7/IL-24 protein comprises an amino acid sequence of SEQ ID NO: 3. In embodiments, the composition further comprises a pharmaceutically acceptable excipient. In embodiments, the composition is for use in preventing or treating bone metastasis in a subject with cancer. In embodiments, the cancer is prostate cancer. In embodiments, he prostate cancer comprises cancer cells having an increased expression of one or more of Mcl-1, RANKL, Bcl-2, Bcl-xL, and Akt, relative to normal prostate cells.

In some aspects, the present disclosure provides a use of a composition described herein in the manufacture of a medicament. In embodiments, the medicament is for the prevention or treatment of bone metastasis in a subject with cancer. In embodiments, the medicament is for use in accordance with a method described herein.

In some aspects, the present disclosure provides a kit. In embodiments, the kit comprises an MDA-7/IL-24 protein and one or both of an Mcl-1 inhibitor and a PI3K inhibitor described herein.

### BRIEF DESCRIPTION OF THE DRAWINGS

FIG. 1A illustrates a schematic diagram of an example method for producing a His-tagged MDA-7/IL-24 protein.

FIG. 1B illustrates an example western blot confirming production of MDA-7/IL-24 protein.

FIG. 1C is a bar graph illustrating example results for effects of MDA-7/IL-24 protein on proliferation of PC3-ML cells, as measured by MTT assay. These results show a significant inhibition in cell proliferation by MDA-7/IL-24 protein.

FIG. 1D illustrates an example western blot showing up-regulation of expression of downstream MDA-7/IL-24 signaling cascade molecules p27, Beclin-1, and BiP/GRP78 in cells treated with MDA-7/IL-24 protein (“MDA-7”) relative to untreated controls (“Control”). EFla represents a loading control.

FIG. 1E is a bar graph illustrating example results for colony formation assays performed with different prostate cancer cells in triplicate, comparing cells treated with MDA-7/IL-24 protein (“MDA-7”) and untreated controls (“Control”). Approximately, 200 cells were plated, treated with His-MDA-7/IL-24, and 2 weeks after treatment they were stained with crystal violet. Numbers of colonies were counted and the data was plotted. Data represents mean±S.D. of two independent experiments; **, P<0.01; ***, P<0.001 versus control.

FIGS. 2A-E illustrate anti-metastatic activity and inhibition of osteoclast differentiation by MDA-7/IL-24 protein. FIG. 2A provides images illustrating example results from an in vivo bone metastasis assay evaluating the effect of His-MDA-7 on bone metastasis development, in which treated mice (“MDA-7”) received 5 mg/kg of His-MDA-7 protein twice a week for 3 weeks (n=5 in each group). FIG. 2B is a bar graph quantifying luciferase intensity in mice of FIG. 2A. FIG. 2C is an example survival plot illustrating the role of MDA-7/IL-24 protein treatment in the enhancement of survival of mice with cancer. Data represents mean±S.D. of two independent experiments: **, P<0.01 versus control. FIGS. 2D and 2E are bar graphs comparing the number of osteoclasts (D) and osteoclast activities (E) in bone marrow cells of control and MDA-7/IL-24-treated mice. Five replicates were done for each group, and data represents mean±S.D. of two independent experiments; *, P<0.05; ***, P<0.001 versus control.

FIGS. 3A-C illustrate example effects of MDA-7/IL-24 protein on signaling cascades in RAW 264.7 cells. Cells were untreated (“C”), or treated with the indicated reagents. Reagents included MDA-7/IL-24 protein (“MDA-7;” 10 μg/mL), receptor activator of nuclear factor kappa-B ligand (“RANKL;” 100 ng/mL), LY294002 (“LY;” 10 μmol/L), and a plasmid encoding a constitutively active form of Akt (“MYR-Akt”). Phosphor-GSK3β, NFATc1, and Mcl-1 expression increased with the over expression of a constitutively active Akt, which were inhibited by treatment with His-MDA-7. EFla was used as a loading control.

FIGS. 4A-D illustrate a combinatorial effect of His-MDA-7 and BI-97D6 on metastasis of prostate cancer to bone and osteoclast differentiation. FIG. 4A provides images illustrating example results from an in vivo bone metastasis assay evaluating the effects of His-MDA-7 and BI-97D6. FIG. 4B is a bar graph of luciferase intensity, quantified in triplicate, for mice imaged as in FIG. 4A. The graph shows significant inhibition in luciferase intensity in MDA-7/IL-24-treated animals. Addition of BI-97D6 further enhanced the inhibitory effects of MDA-7/IL-24 on bone metastasis development. FIGS. 4C and 4D are bar graphs comparing the number of osteoclasts (C) and osteoclast activities (D) in bone marrow cells of control and treated mice. Osteoclasts were stained with a TRAP staining kit and osteoclastic activity was measured using a TRACP enzymatic assay kit. Four replicates were taken for each group. Data represent mean±S.D. of two independent experiments; *, P<0.05; **, P<0.01; ***, P<0.001 versus control.

FIGS. 5A-E illustrate effects of Akt expression on prostate cancer bone metastasis and response to MDA-7/IL-24 and osteoclast differentiation. FIG. 5A illustrates an example western blot comparing phosphor-Akt expression in control PC3-ML cells (“C”) and PC3-ML cells stably overexpressing CA-Akt (“PC3-ML^{Akt}”). FIG. 5B provides images illustrating example results from an in vivo bone metastasis assay, showing that constitutive activation of Akt diminished the inhibitory effects of MDA-7/IL-24 protein on bone metastasis of prostate cancer. FIG. 5C is a bar graph of luciferase intensity for mice imaged as in FIG. 5B. FIGS. 5D and 5E are bar graphs comparing the osteoclast activities (D) and number of osteoclasts (E) in bone marrow cells of control and treated mice. Bone marrow cells from mice were collected and 5×10⁵ cells were induced for osteoclast differentiation. Data represent mean±S.D. of two independent experiments; *, P<0.05; ***, P<0.001 versus control.

FIG. 6 is an example schematic representation of MDA-7/IL-24-mediated inhibition in progression of prostate cancer-derived bone metastasis through modulation of the bone microenvironment.

FIG. 7 is an image illustrating example results from an in vivo bone metastasis assay in mice receiving various doses of MDA-7/IL-24 protein and/or BI-97D6. Mice received intra-cardiac injection of PC3-ML luc cells (1×10⁵) and were treated with recombinant MDA-7 protein (i.v.) as indicated with or without BI-97D6 (1.5 mg/kg-i.p.) (total 6 doses of both, duration of study-3 weeks). Mice were imaged by an IVIS® imaging system.

FIG. 8 provides images of cells illustrating the effect of MDA-7/IL-24 on normal primary mouse bone marrow cells. Primary cells from mouse bone marrow were isolated and cultured in the presence of MDA-7/IL-24 protein. Live-dead assay was performed and images were captured showing no effect of MDA-7/IL-24 on primary bone marrow cells.

FIGS. 9A-C illustrate example effects of MDA-7/IL-24 protein on osteoclast differentiation. FIG. 9A provides images of bone marrow cells from a normal mouse that were induced for osteoclast differentiation without (“Control”) or in the presence of 10 μg/mL MDA-7/IL-24 protein (“MDA-7”). FIGS. 9B-C are bar graphs comparing the number of osteoclasts (B) and osteoclast activities (C) in bone marrow cells of control and treated mice. After 5 days differentiated osteoclasts were stained by TRAP staining kit and osteoclastic activity was measured in triplicates. The number of osteoclasts decreased significantly in His-MDA-7-treated cells. Also, the TRACP enzymatic activity is significantly less in His-MDA-7-treated cells.

FIGS. 10A-C illustrate example effects of MDA-7/IL-24 protein on the osteoclastic gene regulation pattern and growth of RAW 264.7 and DU-145 cells. FIG. 10A shows bar graphs for the expression levels of TRAP, Cathepsin K (CTSK) and Calcitonin R (CTR) genes, as measured by RQ-PCR in RNA isolated after 5 days of treatment with one or both of MDA-7/IL-24 protein (10 μg/mL) or RANKL (100 ng/mL), compared to untreated control cells. FIGS. 10B-C provide bar graphs of results for proliferation assays in RAW 264.7 cells (B) and DU-145 cells (C) treated with MDA-7/IL-24 protein compared to untreated controls. 2000 cells were plated in 96-well plates in quadruplicates and treated with His-MDA-7 (10 μg/ml) and MTT assays were done after 5 days. Data represents mean±S.D. of two independent experiments; **, P<0.01; ***, P<0.001 versus control

FIGS. 11A-C are bar graphs illustrating densitometric quantification of results shown in FIGS. 3A-3C, respectively, for p-Akt/Akt, p-GSK3β/GSK3β, NFATc1/EF1α, and Mcl-1/EF1α (n=3). Statistical analysis was done by unpaired t-test. a, P<0.05 versus control; b, P<0.05 versus RANKL (A), LY (B), and MYR-Akt (C); ns=not significant.

FIGS. 12A-B are bar graphs illustrating example effects of Mcl-1 inhibitors and MDA-7/IL-24 protein on osteoclast differentiation. FIG. 12A illustrates results for two Mcl-1 inhibitors (BI-97C1 and BI-97D6). Bone marrow cells from mice were collected and 5×10⁵ cells were induced for osteoclast differentiation in quadruplicate in the presence or absence of the indicated doses of inhibitors. FIG. 12B illustrates results for primary bone marrow cells that were induced for osteoclast differentiation treated with MDA-7/IL-24 and/or BI-97D6. Data represents mean±S.D. of two independent experiments; *, P<0.05; ***, P<0.001 versus control.

FIGS. 13A-B are bar graphs illustrating example effects of Akt and Mcl-1 expression on osteoclast differentiation. FIG. 13A illustrates the numbers of osteoclasts obtained when primary cells from mouse bone marrow were isolated and cultured in triplicate in the presence of MCSF (10 ng/mL) and RANKL (100 ng/mL) and treated with MDA-7/IL-24 protein (10 μg/mL) and/or an indicated amount of PI3K inhibitor LY294002, compared to untreated control. FIG. 13B illustrates osteoclastic activity (measured in triplicate) in RAW 264.7 cells that were stably transfected with a constitutive Akt (CA-Akt), dominant negative Akt (DN-Akt), or Mcl-1 overexpression plasmid (MCL1), and induced to undergo osteoclast differentiation.

FIG. 14 illustrates example western blots showing downstream signaling cascades in Akt overexpressing stable PC3-ML cells (“Cl. 1” and “Cl. 2”) compared to control cells (“C”). Clone 1 (“Cl. 1”) showed significant upregulation in p-Akt, p-GSK3β, and cyclin-D1 expression.

FIG. 15 is an image of mice that received an intra-cardiac injection of PC3-ML luciferase cells (1×10⁵) and untreated (control) or treated with six doses of MDA-7/IL-24 protein (5 mg/kg). Mice were imaged by an IVIS® imaging system.

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

Section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entireties for any purpose.

### Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); and Sambrook and Green, Molecular Cloning: A Laboratory Manual, 4th Edition (2012). Methods, devices and materials similar or equivalent to those described herein can be used in the practice of this invention. The following definitions are provided to facilitate understanding of certain terms used frequently herein and are not meant to limit the scope of the present disclosure.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

It is noted that, as used herein and in the appended claims, the singular forms “a,” “an,” and “the” include plural referents unless the context clearly dictates otherwise. It is further noted that the claims may be drafted to exclude any optional element. As such, this statement is intended to serve as support for the recitation in the claims of such exclusive terminology as “solely,” “only,” and the like in connection with the recitation of claim elements, or use of a “negative” limitations, such as “wherein [a particular feature or element] is absent,” or “except for [a particular feature or element],” or “wherein [a particular feature or element] is not present (included, etc.) . . . .”

The terms “nucleic acid,” “nucleic acid molecule,” “nucleic acid oligomer,” “oligonucleotide,” “nucleic acid sequence,” “nucleic acid fragment,” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i. e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide,” and “protein” are used interchangeably herein to refer to a polymer of amino acid residues, of any length. The polymer may be linear or branched, it may comprise modified amino acids, and it may be interrupted by non amino acids. The terms also encompass an amino acid polymer that has been modified; for example, by disulfide bond formation, glycosylation, lipidation, acetylation, phosphorylation, or any other manipulation, such as conjugation with a labeling component. A “fusion protein” refers to a chimeric protein encoding two or more separate protein sequences that are recombinantly expressed as a single moiety.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST or the like). In embodiments, sequences that are “substantially identical” are at least 80%, 90%, 95%, 99%, or more identical. In the case of nucleic acids, percent identity may also refer to, or may be applied to, the complement of a test sequence. As described below, the preferred algorithms can account for gaps and the like. In embodiments, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions as compared to the reference sequence (which does not comprise the additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison (e.g., with respect to the reference sequence), and multiplying the result by 100 to yield the percentage of sequence identity. Programs for determining sequence identify are known to those skilled in the art, and include, without limitation, BLAST (as noted above, optionally using default parameters), the Needleman-Wunsch algorithm (see e.g. the EMBOSS Needle aligner available at www.ebi.ac.uk/Tools/psa/emboss_needle/nucleotide.html, optionally with default settings).

The terms “MDA-7,” “IL-24,” or “MDA-7/IL-24” refer to a protein (including homologs, isoforms, and functional fragments thereof) with MDA-7 activity. The term includes any recombinant or naturally-occurring form of MDA-7 or variants, homologs, or isoforms thereof that maintain MDA-7 activity (e.g. within at least 30%, 40%, 50%, 60%, 70%, 80%, 90%, 95%, or 100% activity compared to wild-type MDA-7). In embodiments, the variants, homologs, or isoforms have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring MDA-7 protein. In embodiments, the MDA-7 protein is substantially identical to the protein identified by Accession No. NP_006841 or a variant or homolog having substantial identity thereto. In embodiments, the MDA-7 protein is substantially identical to the protein identified by UniProt Q13007 or a variant or homolog having substantial identity thereto. In embodiments, the IL-24 gene is substantially identical to the nucleic acid sequence set forth in RefSeq (mRNA) NM_006850, or a variant or homolog having substantial identity thereto. In embodiments, the IL-24 gene is substantially identical to the nucleic acid sequence set forth in Ensembl reference number ENSG00000162892, or a variant or homolog having substantial identity thereto. In embodiments, the amino acid sequence or nucleic acid sequence is the sequence known at the time of filing of the present application. In embodiments, the protein is a mature form of MDA-7, in which a signal sequence at the N-terminus of a precursor form of the protein is absent. The mature form can be produced post-translationally from a precursor form containing a signal sequence, or can be translated directly from a polynucleotide encoding the mature form without a signal sequence N-terminal with respect to the sequence of the mature MDA-7. In embodiments, the MDA-7/IL-24 protein comprises SEQ ID NO: 2, or variants, homologs, or isoforms thereof that maintain MDA-7 activity. In embodiments, the MDA-7/IL-24 protein comprises SEQ ID NO: 3, or variants, homologs, or isoforms thereof that maintain MDA-7 activity. In embodiments, the MDA-7/IL-24 protein does not comprise the first 49 amino acids of SEQ ID NO: 2. In embodiments, the MDA-7/IL-24 protein comprises SEQ ID NO: 4, or variants, homologs, or isoforms thereof that maintain MDA-7 activity.

One of skill in the art will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

Certain amino acids may be substituted for other amino acids in a protein structure without appreciable loss of tumoricidal effects. Since it is the interactive capacity and nature of a protein that defines that protein's biological functional activity, certain amino acid substitutions can be made in a protein sequence, and, of course, in its DNA encoding sequence, and nevertheless obtain a protein with like properties. It is thus contemplated that various changes may be made in the polypeptide sequences of the present disclosure, or corresponding DNA sequences which encode said polypeptides, while retaining at least some of their biological activity. Such biological activity can be assessed by various techniques, such as for instance assays described in the examples herein.

The term “purified,” when applied to a nucleic acid or protein, denotes that the nucleic acid or protein is essentially free of one or more other cellular components with which it is associated in the natural state or in a whole cell lysate. It can be, for example, in a homogeneous state or in a mixture with one or more other compounds, and may be in either a dry or aqueous solution. For example, an MDA-7/IL-24 protein may be purified from a cell lysate, then combined with one or more other agents (e.g., an Mcl-1 inhibitor and/or a PI3K inhibitor). As such, compositions comprising a purified MDA-7/IL-24 protein may comprise compounds in addition to the MDA-7/IL-24 protein, but will generally lack or be reduced in one or more impurities present in a lysate or media from which an MDA-7/IL-24 protein is initially isolated. Purity and homogeneity are typically determined using analytical chemistry techniques such as polyacrylamide gel electrophoresis or high performance liquid chromatography. A protein that is the predominant species present in a preparation is substantially purified.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer is a cancer that metastasized to bone. In embodiments, the cancer is prostate cancer, such as prostate cancer-derived bone metastasis.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., prostate, which site is referred to as a primary tumor, e.g., primary prostate cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if prostate cancer metastasizes to the bone, the secondary tumor at the site of the bone consists of abnormal prostate cells and not abnormal bone cells. The secondary tumor in the bone is referred to as a metastatic bone cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the bone.

The terms “osteoclast differentiation” and “osteoclast formation” refer to a process in which osteoclast precursor (progenitor) cells are recruited from hematopoietic compartments, and then proliferate and differentiate toward mature osteoclasts. During this multi-step differentiation process, postmitotic osteoclast precursors progressively express osteoclast-associated markers, such as cathepsin-K, MMP-9, calcitonin receptor and tartrate-resistant acid phosphatase (TRAP), while losing some of their macrophage characteristics. Then, mononuclear preosteoclasts fuse together to form multinucleated giant cells. Terminal osteoclast differentiation eventually leads to active bone-resorbing cells. Once formed, the osteoclast may be referred to as large osteoclasts, which are typically those characterized by several nuclei (e.g., up to several dozens) or small osteoclasts containing few nuclei (e.g., as few as three).

As used herein, a “subject” can be a mammal such as a non-primate (e.g., cows, pigs, horses, cats, dogs, rats, etc.) or a primate (e.g., monkey and human). In embodiments, the subject is a human. In embodiments, the subject is a mammal (e.g., a human) having or potentially having a cancer, such as a metastatic cancer, described herein. In embodiments, the subject is a mammal (e.g., a human) at risk of developing a cancer, such as a metastatic cancer, described herein.

“Treating” or “treatment” as used herein broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, delay or slowing of disease progression, amelioration or palliation of the disease state, diminishment of the reoccurrence of disease, and remission, whether partial or total and whether detectable or undetectable. In other words, “treatment” as used herein includes any cure or amelioration of a disease. Treatment may relieve the disease's symptoms fully or partially remove the disease's underlying cause, shorten a disease's duration, or do a combination of these things. In the case of cancer, treatment may include slowing, halting, or reversing cancer cell multiplication (e.g., as in growth of a tumor, as measured by tumor size or a rate of change thereof).

“Preventing” as used herein refers to a decrease in the occurrence or incidence of one or more disease symptoms in a patient. Prevention may be complete (no detectable symptoms) or partial, such that fewer symptoms are observed than would likely occur absent treatment. Prevention includes prophylactic treatment.

The length of treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prevention may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, administering a composition of the present disclosure both treats a cancer of a subject (e.g., metastatic bone cancer), and prevents further disease systems (e.g., bone metastases).

The compositions described herein can be used in combination with one another, with other active agents known to be useful in treating a cancer such as anti-cancer agents. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cancer cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

As used herein, the term “administering” encompasses oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). In embodiments, “administering” a protein or a composition comprising the protein refers to administering the protein itself (e.g., an MDA-7/IL-24 protein), rather than a polynucleotide encoding the protein.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

A “combined synergistic amount” as used herein refers to the sum of a first amount (e.g., an amount of MDA-7/IL-24 recombinant protein) and a second amount (e.g., an amount of an Mcl-1 inhibitor), that results in a synergistic effect (i.e. an effect greater than an additive effect). Therefore, the terms “synergy,” “synergism,” “synergistic,” “combined synergistic amount,” and “synergistic therapeutic effect” which are used herein interchangeably, refer to a measured effect of compounds administered in combination where the measured effect is greater than the sum of the individual effects of each of the compounds administered alone as a single agent.

In embodiments, a combined synergistic amount is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the first amount (e.g., MDA-7/IL-24 protein) when used separately from the second amount (e.g., Mcl-1 inhibitor). In embodiments, a combined synergistic amount is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the second amount (e.g., Mcl-1 inhibitor) when used separately from the first amount (e.g., MDA-7/IL-24 protein).

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor that binds to a target protein). In embodiments, inhibition refers to a reduction of activity of a target protein from an indirect interaction (e.g., an inhibitor that binds to a protein that activates a target protein, thereby preventing target protein activation). “Mcl-1 inhibitors” include compounds that negatively affect (e.g. decreases) the activity or function of Mcl-1 or other signaling pathway components (e.g., proteins, genes) involved in the Mcl-1 signaling pathway relative to the activity or function of Mcl-1 or signaling pathway components (e.g., proteins, genes) involved in the Mcl-1 signaling pathway in the absence of the inhibitor. In embodiments, an Mcl-1 inhibitor is an agent that directly binds to an inhibits the activity of Mcl-1.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components. For example, binding of a Mcl-1 with a compound as described herein (e.g., Mcl-1 inhibitor) may reduce the level of a product of the Mcl-1 catalyzed reaction or the level of a downstream derivative of the product, or binding may reduce the interactions between Mcl-1 or an Mcl-1 reaction product and downstream effectors or signaling pathway components, resulting in changes in cell growth, proliferation, or survival.

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

### Methods

In various aspects, the present disclosure provides methods of preventing metastasis to bone in a subject with cancer, and methods of treating bone metastasis in a subject with cancer. In embodiments, the methods comprise administering an effective amount of a composition comprising an MDA-7/IL-24 protein to the subject. The cancer of the subject may or may not have metastasized prior to the administering. In embodiments, bone metastasis is prevented in a subject in which bone metastasis has not yet been detected. In embodiments, the subject has one or more bone metastases, and preventing metastasis to bone comprises preventing further bone metastases. In embodiments, the subject has one or more bone metastases, and treating the bone metastasis comprises slowing, halting, or reversing growth of one or more such metastases. In embodiments, the subject is at risk of developing metastatic cancer or at risk of developing additional bone metastases, and preventing metastasis to bone comprises reducing the risk or incidence of such metastases, relative to the risk or expected incidence in the absence of such preventing.

In embodiments, the MDA-7/IL-24 protein is a recombinantly produced protein. Various methods for recombinant protein production are available. Non-limiting examples include in vitro translation, and production in host cells. In embodiments, host cells used to produce a recombinant protein express a polynucleotide encoding the protein or a precursor thereof. Such polynucleotides may be transiently transfected into the host cell (e.g., by way of a plasmid or a viral vector), or may be stably integrated into the genome of the host cell. A variety of suitable host cells are available. In embodiments, the host cells are immortalized primary human fetal astrocyte (IM-PHFA) cells.

In embodiments, producing the MDA-7/IL-24 protein comprises transfecting a host cell with a virus comprising a polynucleotide encoding the MDA-7/IL-24 protein. In embodiments, the virus is an adenovirus. A variety of suitable adenoviruses are available. Non-limiting examples of adenoviruses that may be used in the production of an MDA-7/IL-24 protein include those described in WO2018089995A1, WO2017062708A1, US20180243382A1, US20160008413A1, and Dash et al, Cancer Res 2014; 74:563-74.

In embodiments, the MDA-7/IL-24 protein is a purified protein. A variety of methods for purification are available, which depend, in part, on the method of production. For example, if a host cell is used and the protein is not secreted by the host cell, purification may comprise the step of lysing the host cells to release the protein. In embodiments, the MDA-7/IL-24 protein is secreted from a host cell, and purification may comprises purification without cell lysis. The mode of purification will also depend on the nature of the protein produced. For example, the MDA-7/IL-24 protein produced by the host cell can comprise additional elements, such as a protein tag to facilitate purification (e.g., a His, FLAG, or HA tag). A protein tag facilitates purification using a cognate binding partner (e.g., nickel in the case of a His tag), which may be adhered to a substrate. In embodiments, an MDA-7/IL-24 protein initially produced with a purification tag is treated to remove the tag before administration to a subject.

In embodiments, the MDA-7/IL-24 protein produced by a host cell does not comprise a purification tag. In such cases, purification may comprise purification using reagents that bind to the MDA-7/IL-24 protein (e.g., antibodies adhered to a substrate). In embodiments, purification comprises removal of components of a media or lysate other than the MDA-7/IL-24 protein. For example, a lysate or cellular suspension can be centrifuged to produce a pellet of cells or cellular debris, and the supernatant separated to a different container, thereby purifying the MDA-7/IL-24 protein by separation of such cells or cellular debris.

In embodiments, the MDA-7/IL-24 protein retains a biological activity. As a cytokine and a member of the IL-10 cytokine gene family, MDA-7/IL-24 natively signals through receptor dimers consisting of an R1 type receptor and an R2 type receptor (IL-20R1 and IL-20R2; IL-22R1 and IL-20R2) or a unique receptor pair consisting of two R1 type receptors (IL-20R1 and IL-22R1) in order to activate downstream signaling events. Assays for measuring such activities are available (see, e.g., WO2018089995A1). In embodiments, an MDA-7/IL-24 protein is a variant, homolog, or isoform that retains at least 50%, 60%, 70%, 80%, 85%, 90%, 95%, or more of the biological activity of an MDA-7/IL-24 protein of SEQ ID NO: 2 or SEQ ID NO: 3. In embodiments, the MDA-7/IL-24 protein retains at least 80% of the biological activity of an MDA-7/IL-24 protein of SEQ ID NO: 3. In embodiments, the MDA-7/IL-24 protein retains at least 90% of the biological activity of an MDA-7/IL-24 protein of SEQ ID NO: 3. In embodiments, the MDA-7/IL-24 protein is capable of activating an IL-20/IL-22 receptor complex of a cancer cell of the subject, or of a reference cell line (e.g. DU-145 cells).

In embodiments, the MDA-7/IL-24 protein is a precursor protein, comprising a signal sequence. For example, the MDA-7/IL-24 protein may comprise an amino acid sequence of SEQ ID NO: 2. In embodiments, the MDA-7/IL-24 protein comprises an amino acid sequence that is at least 80%, 85%, 90%, 95%, or more identical to SEQ ID NO: 2. In embodiments, the MDA-7/IL-24 protein consists or consists essentially of a polypeptide of SEQ ID NO: 2.

In embodiments, the MDA-7/IL-24 protein is a mature MDA-7/IL-24 protein. In embodiments, the MDA-7/IL-24 protein comprises an amino acid sequence of SEQ ID NO: 3. In embodiments, the MDA-7/IL-24 protein comprises an amino acid sequence that is at least 80%, 85%, 90%, 95%, or more identical to SEQ ID NO: 3. In embodiments, the MDA-7/IL-24 protein consists or consists essentially of a polypeptide of SEQ ID NO: 3.

In embodiments, the MDA-7/IL-24 protein is a truncated form of MDA-7/IL-24 protein that retains biological activity. For example, the MDA-7/IL-24 protein may lack the first 54 amino acids of SEQ ID NO: 3. In embodiments, the MDA-7/IL-24 protein comprises SEQ ID NO: 4. In embodiments, the MDA-7/IL-24 protein comprises an amino acid sequence that is at least 80%, 85%, 90%, 95%, or more identical to SEQ ID NO: 4. In embodiments, the MDA-7/IL-24 protein consists or consists essentially of a polypeptide of SEQ ID NO: 4.

In embodiments, administering a composition comprising the MDA-7/IL-24 protein comprises administering to a target tissue, such as to a tumor, a site from which a tumor has been surgically removed, and/or to a bone of a subject. In embodiments, administering to the target tissue comprises injection into or adjacent to the target tissue, or topical application to the target tissue. In embodiments, the composition is delivered distally to the target tissue, but is formulated to traffic the MDA-7/IL-24 protein to the target tissue. In embodiments, a moiety that traffics to a particular tissue, such as a cancer tissues and/or a bone tissue, is complexed with the MDA-7/IL-24 protein. Complexing can be directly with the targeting moiety, such as a covalent or non-covalent interaction between the MDA-7/IL-24 protein and the targeting moiety. Complexing can be indirect, such that the MDA-7/IL-24 protein and the targeting moiety are separated by one or more other molecules joining the two, via covalent or non-covalent interactions. In general, a targeting moiety is a moiety able to bind to or otherwise associate with a biological entity (e.g., a membrane component, a cell surface receptor, cell specific membrane antigen, or the like), with a higher affinity than one or more non-target biological entity (e.g., cell surface components of one or more different tissues). A targeting moiety typically allows a cargo (e.g., an MDA-7/IL-24 protein) to become localized at a particular targeting site to a higher degree than elsewhere in the body of the subject, or to a higher degree at the target site than would be accomplished in the absence of the targeting moiety. Non-limiting examples of targeting moieties include antibodies, antigen-binding antibody fragments, aptamers, peptides, hormones, growth factors, ligands (e.g., receptor ligands), small molecules, and the like. Illustrative examples of targeting moieties that traffic to bone are described in US20120028350A1, US20160052968A1, US20040038946A1, and US20180208650A1.

In embodiments, administration comprises ultrasound-targeted microbubble-destruction (UTMD), allowing for directed delivery of MDA-7/IL-24 protein to a target tissue. For example, a composition of the present disclosure may be complexed with microbubbles, administered intravenously, then released at a target tissue by applying ultrasound at the target tissue. US20180243382A1 provides an illustrative example of microbubble delivery technology.

In embodiments, bone metastasis of any of a variety of cancers is treated or prevented. Some cancers have a higher propensity to metastasize to bone. Examples include, without limitation, prostate cancer, breast cancer, lung cancer, kidney cancer, and thyroid cancer. In embodiments, the cancer is a prostate cancer. Various gene expression signatures can be used to distinguish cancer cells from non-cancer cells, cancers of one tissue from cancers of another tissue, and metastatic cancers from non-metastatic cancers. In embodiments, the cancer is a prostate cancer having an increased expression of one or more of Mcl-1, RANKL, Bcl-2, Bcl-xL, and Akt, relative to normal prostate cells, such as a reference prostate cell line or non-cancerous prostate cells of the subject with prostate cancer.

In embodiments, the composition comprising the MDA-7/IL-24 protein further comprises one or more additional agents, or is co-administered with one or more additional agents. In embodiments, the one or more additional agents is an Akt inhibitor, an Mcl-1 inhibitor, or a combination thereof. In embodiments, the one or more additional agents is a phosphoinositide 3-kinase (PI3K) inhibitor, an Mcl-1 inhibitor, or a combination thereof. In embodiments, the additional agent is a PI3K inhibitor (e.g., LY294002). In embodiments, the additional agent is an Mcl-1 inhibitor (e.g., BI-97D6).

A variety of Akt inhibitors are available, which may be subdivided into several classes. A first class contains ATP competitive inhibitors of Akt and includes compounds such as CCT128930 and GDC-0068, which inhibit Akt2 and Akt1. This category also includes the pan-Akt kinase inhibitors such as GSK2110183 (afuresertib), GSK690693, and AT7867. A second class contains lipid-based Akt inhibitors that act by inhibiting the generation of PIP3 by PI3K. This mechanism is employed by phosphatidylinositol analogs such as Calbiochem Akt Inhibitors I, II and III or other PI3K inhibitors such as PX-866. This category also includes compounds such as Perifosine (KRX-0401) (Aetema Zentaris/Keryx). A third class contains a group of compounds called pseudosubstrate inhibitors. These include compounds such as AKTide-2 T and FOXO3 hybrid. A fourth class consists of allosteric inhibitors of AKT kinase domain, and include compounds such as MK-2206 (8-[4-(1-aminocyclobutyl)phenyl]-9-phenyl-2H-[1,2,4]triazolo[3,4-f][1,6]naphthyridin-3-one; dihydrochloride) (Merck & Co.) (see, e.g., U.S. Pat. No. 7,576,209). A fifth class includes antibodies, such as GST-anti-Akt1-MTS. A sixth class comprises compounds that interact with the PH domain of Akt, and includes Triciribine and PX-316. Other compounds that act as AKT inhibitors include, for example, GSK-2141795 (GlaxoSmithKline), VQD-002, miltefosine, AZD5363, GDC-0068, RX-0201 (an antisense oligonucleotide), PBI-05204, SRI 3668, and API-1.

A variety of PI3K inhibitors are available, some of which are noted above. Additional examples include, but are not limited to, wortmannin (an irreversible inhibitor of PI3K), demethoxyviridin (a derivative of wortmannin), LY294002 (a reversible inhibitor of PI3K); BKM120 (Buparlisib), Idelalisib (a PI3K Delta inhibitor), duvelisib (IPI-145, an inhibitor of PI3K delta and gamma), alpelisib (BYL719, an alpha-specific PI3K inhibitor), TGR 1202 (also known as RP5264, an oral PI3K delta inhibitor), copanlisib (BAY 80-6946, an inhibitor PI3Kα,δ), BEZ235, RP6530, TGR 1201, SF1126, INK1117, GDC-0941, XL147 (SAR245408), XL765 (SAR245409), Palomid 529, GSK1059615, ZSTK474, PWT33597, IC87114, TGI00-115, CAL263, RP6503, PI-103, GNE-477, CUDC-907, AEZS-136, GDC-0980, and GDC-0032. In embodiments, the PI3K inhibitor is LY294002.

A variety of Mcl-1 inhibitors are available. Non-limiting examples of Mcl-1 inhibitors include BI97C10, BI112D1, gossypol (AT-101, Ascenta Therapeutics), obatoclax (GX15-070, Cephalon), MG-132, MIM1, sabutoclax (BI97C1, Oncothyreon), and TW-37. Further examples of Mcl-1 inhibitors are disclosed in Varadarajan et al. (Cell Death Differ. 2013 November; 20(11): 1475-1484), Tanaka et al. (J Med Chem 56(23):9635-9645 (2013)), Friberg, et al. (J Med Chem 56(1): 15-30 (2013)), US20150045357A1, US20150051249A1, US20130035304A1, US20090054402A1, and US20110112112A1. In embodiments, the Mcl-1 inhibitor is BI-97D6.

In embodiments, the effective amount is an amount of the composition effective to prevent or treat bone metastasis. In embodiments, the effective amount comprises an amount of the composition (or a component thereof, e.g. the MDA-7/IL-24 protein) that is substantially non-toxic to primary bone marrow cells or normal primary human prostate epithelial cells. In embodiments, an amount of a compound is substantially non-toxic to primary bone marrow cells when the amount induces no increased cell death relative to the absence of the compound, or any increase in cell death is less than 20%, 15%, 10%, 5%, or less relative to the absence of the compound. Toxicity effects can be measured, for example, using commercially available live-dead cell staining assays.

In embodiments, the effective amount comprises an amount of the composition (or a component thereof, e.g. the MDA-7/IL-24 protein) that is an amount that inhibits osteoclast differentiation. In embodiments, inhibition of osteoclast differentiation comprises a reduction in the number of bone marrow cells that differentiate into osteoclasts by at least 5%, 10%, 15%, 20%, 25%, 30%, 35%, 40%, 50%, 60%, 70%, or more. In embodiments, differentiation of bone marrow cells to osteoclasts is reduced by at least 25%. Effects on osteoclast differentiation can be measured, for example, by comparing treated and untreated cells in culture, or by comparing the number of osteoclasts in a bone marrow sample from a treated subject to the number of osteoclasts in a comparable bone marrow sample from an untreated subject. Examples of assays for measuring effects on osteoclast differentiation, including effects on the number of osteoclasts and effects on osteoclast activity, are described herein.

In embodiments, the amount of MDA-7/IL-24 protein administered to a subject is one or more doses of at least 0.5 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 6 mg/kg, 7 mg/kg, 8 mg/kg, 9 mg/kg, 10 mg/kg, 15 mg/kg, or 20 mg/kg. In embodiments, the amount of MDA-7/IL-24 protein administered to a subject is one or more doses of between 0.5 mg/kg and 20 mg/kg, between 1 mg/kg and 10 mg/kg, or between 2.5 mg/kg and 7.5 mg/kg. In embodiments, the amount of MDA-7/IL-24 protein administered to a subject is about 5 mg/kg. In embodiments, the effective amount is administered in a single administration, or in a plurality of doses (e.g., 2, 3, 4, 5, 6, 7, 8, 9, 10, 15, or more doses). A plurality of doses can be administered at regular or irregular intervals, such as one or more times a week (e.g., 2, 3, 4, 5, or more times a week), once every period of weeks (e.g., weekly, or every 2, 3, 4, 5, or more weeks), or once every period of days (e.g., daily, or every 2, 3, 4, 5, or more days). In embodiments, the composition is administered at intervals until a desired therapeutic result is achieved (e.g., absence of bone metastases for a period of time).

In embodiments, the composition comprises an amount of an Mcl-1 inhibitor (e.g., BI-97D6), such as at least about 0.1 mg/kg, 0.2 mg/kg, 0.3 mg/kg, 0.4 mg/kg, 0.5 mg/kg, 0.75 mg/kg, 1 mg/kg, 1.25 mg/kg, 1.5 mg/kg, 2 mg/kg, 3 mg/kg, 4 mg/kg, or 5 mg/kg inhibitor is administered to the subject. In embodiments, between 0.1 mg/kg and 5 mg/kg, between 0.2 mg/kg and 3 mg/kg, or between 0.5 mg/kg and 2 mg/kg are administered to the subject. In embodiments, the composition comprises the Mcl-1 inhibitor at a dose of about 1.5 mg/kg. In embodiments, the Mcl-1 inhibitor is administered separately from the MDA-7/IL-24 protein, but according to the same dosing schedule.

### Compositions

In various aspects, the present disclosure provides compositions for use in or produced by methods described herein, and for use in the manufacture of a medicament for use in methods described herein, including with respect to any of the various embodiments noted above. Compositions of the disclosure can comprise any one or more of the elements described herein. In embodiments, the present disclosure provides compositions for use in preventing metastasis to bone in a subject with cancer, and compositions for use in treating bone metastasis in a subject with cancer.

In embodiments, the composition comprises an MDA-7/IL-24 protein and one or more of an Mcl-1 inhibitor, an Akt inhibitor, or PI3K inhibitor. In embodiments, the composition comprises an MDA-7/IL-24 protein and one or both of an Mcl-1 inhibitor and a PI3K inhibitor. In embodiments, the MDA-7/IL-24 protein is an MDA-7/IL-24 protein described herein, such as with regard to the various methods of the disclosure. In embodiments, the Akt inhibitor is an Akt inhibitor described herein, such as with regard to the various methods of the disclosure. In embodiments the PI3K inhibitor is a PI3K inhibitor described herein, such as with regard to the various methods of the disclosure (e.g., LY294002). In embodiments, the Mcl-1 inhibitor is an Mcl-1 inhibitor described herein, such as with regard to the various methods of the disclosure (e.g., BI-97D6). In embodiments, the composition is a pharmaceutical composition. In embodiments, the composition comprises a pharmaceutically acceptable excipient.

### Kits

In various aspects, the present disclosure provides kits for use in any of the methods described herein, including with respect to any of the various embodiments noted above. In embodiments, the kit comprises one or more compositions described herein. Elements of a kit can be provided in any amount and/or combination (such as in the same kit or same container). In some embodiments, kits comprise additional agents for use according to the methods described herein. In embodiments, the kit comprises an MDA-7/IL-24 protein and one or more of an Mcl-1 inhibitor, an Akt inhibitor, or PI3K inhibitor. In embodiments, the kit comprises an MDA-7/IL-24 protein and one or both of an Mcl-1 inhibitor and a PI3K inhibitor. In embodiments, the MDA-7/IL-24 protein is an MDA-7/IL-24 protein described herein, such as with regard to the various methods of the disclosure. In embodiments, the Akt inhibitor is an Akt inhibitor described herein, such as with regard to the various methods of the disclosure. In embodiments the PI3K inhibitor is a PI3K inhibitor described herein, such as with regard to the various methods of the disclosure (e.g., LY294002). In embodiments, the Mcl-1 inhibitor is an Mcl-1 inhibitor described herein, such as with regard to the various methods of the disclosure (e.g., BI-97D6).

### SEQUENCES

(nucleotide sequence encoding an MDA-7/IL-24
protein)
SEQ ID NO: 1
atgaattttcaacagaggctgcaaagcctgtggactttagccagaccctt
ctgccctcctttgctggcgacagcctctcaaatgcagatggttgtgctcc
cttgcctgggttttaccctgcttctctggagccaggtatcaggggcccag
ggccaagaattccactttgggccctgccaagtgaagggggttgttcccca
gaaactgtgggaagccttctgggctgtgaaagacactatgcaagctcagg
ataacatcacgagtgcccggctgctgcagcaggaggttctgcagaacgtc
tcggatgctgagagctgttaccttgtccacaccctgctggagttctactt
gaaaactgttttcaaaaactaccacaatagaacagttgaagtcaggactc
tgaagtcattctctactctggccaacaactttgttctcatcgtgtcacaa
ctgcaacccagtcaagaaaatgagatgttttccatcagagacagtgcaca
caggcggttcctgctattccggagagcatttaaacagttggacgtagaag
cagctctgaccaaagcccttggggaagtggacattcttctgacctggatg
cagaaattctacaagctctga
(amino acid sequence of an MDA-7/IL-24 protein)
SEQ ID NO: 2
MNFQQRLQSLWTLARPFCPPLLATASQMQMVVLPCLGFTLLLWSQVSGAQ
GQEFHFGPCQVKGVVPQKLWEAFWAVKDTMQAQDNITSARLLQQEVLQNV
SDAESCYLVHTLLEFYLKTVFKNYHNRTVEVRTLKSFSTLANNFVLIVSQ
LQPSQENEMFSIRDSAHRRFLLFRRAFKQLDVEAALTKALGEVDILLTWM
QKFYKL
(amino acid sequence of an MDA-7/IL-24 protein)
SEQ ID NO: 3
QGQEFHFGPCQVKGVVPQKLWEAFWAVKDTMQAQDNITSARLLQQEVLQN
VSDAESCYLVHTLLEFYLKTVFKNYHNRTVEVRTLKSFSTLANNFVLIVS
QLQPSQENEMFSIRDSAHRRFLLFRRAFKQLDVEAALTKALGEVDILLTW
MQKFYKL
(amino acid sequence of an MDA-7/IL-24 protein)
SEQ ID NO: 4
ESCYLVHTLLEFYLKTVFKNYHNRTVEVRTLKSFSTLANNFVLIVSQLQP
SQENEMFSIRDSAHRRFLLFRRAFKQLDVEAALTKALGEVDILLTWMQKF
YKL

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entireties for all purposes.

### EXAMPLES

### Example 1: Recombinant MDA-7/IL-24 Suppresses Prostate Cancer Bone Metastasis

Prostate cancer (PC) is a principal cause of cancer-associated morbidity in men. Although 5-year survival of patients with localized PC approaches 100 percent, survival decreases precipitously after metastasis. Bone is the preferred site for disseminated PC cell colonization, altering the equilibrium of bone homeostasis resulting in weak and fragile bones. Currently, no curative options are available for PC bone metastasis. The therapeutic properties of purified MDA-7/IL-24 recombinant protein have not been evaluated in PC or in metastasis.

In this example, MDA-7/IL-24 delivered as a recombinant protein was tested for anti-cancer properties. Using bone metastasis experimental models, animals treated with recombinant MDA-7/IL-24 had significantly less metastatic lesions in their femurs as compared to controls. The inhibitory effects of MDA-7/IL-24 on bone metastasis resulted from PC-selective killing and inhibition of osteoclast differentiation, which plays a role in bone resorption. Gain- and loss-of-function genetic approaches indicated that pro-survival Akt and Mcl-1 pathways are critically important in the anti-bone metastatic activity of MDA-7/IL-24. An Mcl-1 small molecule inhibitor synergized with MDA-7/IL-24 and induced robust anti-bone metastatic activity. These results expand the potential applications of MDA-7/IL-24 as an anti-cancer molecule, and demonstrate that purified recombinant protein is non-toxic in pre-clinical animal models and has profound inhibitory effects on bone metastasis, which can be enhanced further when combined with an Mcl-1 inhibitory small molecule.

Cell Lines, Plasmids, and Mcl-1 Inhibitor: PC3-ML, a metastatic variant of the PC cell line PC3, was grown as described previously (20). This cell line was used to produce PC-induced bone metastasis in athymic male nude mice. DU-145, PC3, RWPE-1 (immortalized normal human prostate epithelial cells), and RAW 264.7 (murine macrophage) cells were obtained from ATCC (American type culture collection, Manassas, Va., USA) and maintained as suggested by the vendor. ARCaP-E and ARCaP-M cell lines and specific media were purchased from Novicure Biotechnology (Birmingham, Ala., USA). For osteoclast differentiation assays, the RAW 264.7 cell line was used. Immortal primary human fetal astrocytes (IM-PHFA) were developed and maintained as described earlier (21). All the cell lines from ATCC and other vendors were purchased during 2012-2016 and authenticated by using STR (short tandem repeat) analysis. Experiments were done with early passage cells. Cells were monitored routinely for contamination including mycoplasma using a mycoplasma detection kit (Sigma-Aldrich, Inc. St. Louis, Mo., USA). Myr-Akt, DN-Akt, and Mcl-1 plasmids were from Addgene (Cambridge, Mass., USA). LY294002, an inhibitor of phosphatidylinositol 3-kinase (PI3 kinase), was purchased from Sigma-Aldrich, Inc. (St. Louis, Mo., USA). Mcl-1 inhibitor, BI-97D6 compound was synthesized and evaluated as described earlier (22,23) and provided by Dr. Maurizio Pellecchia (University California Riverside, Calif.). BI-97D6 inhibits the binding of BH3 peptides to Bel-2, Bcl-xL, and Mcl-1 (22).

Purification of Recombinant His-MDA-7 Protein: IM-PHFA cells were infected with Ad.5-His-mda-7 using a published protocol (24). Cell supernatant was mixed with Ni-NTA (a nickel-nitrilotriacetic) acid slurry to allow binding of MDA-7/IL-24 to the Ni-NTA beads. Twenty-four hours after incubation, the Ni-NTA beads were collected, washed, and the purified MDA-7/IL-24 protein was eluted in imidazole buffer. The protein was validated by western blotting using anti-MDA-7 antibody (Genhunter Corporation, Nashville, Tenn., USA). Biological activity was checked in PC3-ML cells by MTT assay (25).

MTT Cell Proliferation and Clonal Assays: Cell proliferation assays were done as described previously (25). Briefly, 2,000 cells were seeded in each well of a 96-well plate and allowed to attach overnight. Cells were treated with complete media in the presence or absence of different concentrations of MDA-7/IL-24 protein. At appropriate time points, cells were further incubated with MTT (3-(4, 5-di methyl thiazol-2-yl)-2, 5 diphenyl tetrazolium bromide) reagents. Finally, DMSO was applied to dissolve the blue salt and the OD was measured at 560 nm (25). For determining long-term effects, colony formation (cloning) assays were performed as described earlier (26). Briefly, 200 cells were plated and allowed to grow in the presence or absence of MDA-7/IL-24 for an additional 15 days. Culture media was replaced with fresh media containing MDA-7/IL-24 protein once a week, two times in total.

In vivo Metastasis Studies: All animal studies were approved by the Institutional Animal Care and Use Committee (Virginia Commonwealth University). For experimental bone metastasis assays, 6-8-week-old male athymic nude mice (purchased from Harlan, USA) were injected with 1×10⁵ PC3-ML cells stably expressing firefly luciferase gene through an intracardiac route. For determining the therapeutic activity of MDA-7/IL-24 protein, mice received an intravenous injection of recombinant protein (5 mg/kg), one day after implantation of cells. Animals were treated with therapeutics for a total of 6 times (2× a week for first 3 weeks). In combinatorial treatment studies, MDA-7/IL-24 protein (5 mg/kg) and BI-97D6 (1.5 mg/kg) were delivered through tail vein and intraperitoneal route, respectively. Image of the bone region was monitored by a BLI (bioluminescence imaging) method using an IVIS® imaging system (15, 19, 20).

Real Time q-PCR: Total RNA was isolated from cells with the RNA isolation kit from Qiagen (Valencia, Calif., USA). RQ-PCR was performed using TaqMan probes and master mix from Applied Biosystems (Foster City, Calif., USA). Data were analyzed using the graph pad prism software.

Live-Dead Cell Assay: Live and dead cells were observed by confocal laser microscopy (Zeiss, Germany) after staining with live/dead staining reagent (Invitrogen, Carlsbad, Calif., USA) as per the manufacturer's instructions. The images were analyzed by Zeiss software.

Western Blotting: Standard protocols were followed for Western blotting assays (24, 27). The primary antibodies used were pAkt, Akt, pGSK3β, GSK3β, NFATc1, cyclin D1 (Cell Signaling Technology, Danvers, Mass., USA) and EF1α (Abeam, Cambridge, United Kingdom). Appropriate secondary antibodies were purchased from Sigma-Aldrich, Inc. St. Louis, Mo., USA).

Osteoclast Formation Assays: Bone marrow cells were induced for osteoclast differentiation using previously described protocols (28). Briefly, bone marrow cells were cultured in minimal essential medium (α-MEM) with 10% fetal bovine serum with MCSF (10 ng/ml) for 24 hrs. They were subsequently treated with RANKL (100 ng/ml) for 5 days. Cultured cells were fixed and stained for TRAP (Tartarate-resistant acid phosphatase). TRAP staining was performed following the specific protocol provided with the kit (Sigma-Aldrich, Inc. St. Louis, Mo., USA). Multinucleated cells, considered as differentiated osteoclasts, were counted manually under bright field microscope. TRACP enzymatic assays were done as per the manufacturer's instructions (R & D, Minneapolis, Minn., USA).

Statistical Analyses: Statistical analyses were performed using Graph pad prism software. Student's t-test was used to compare the mean differences between groups.

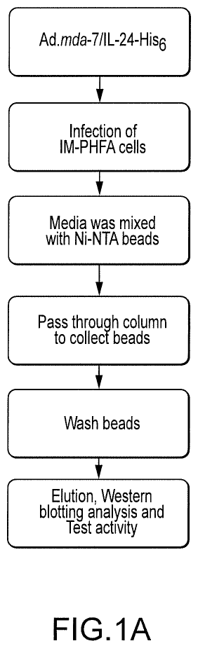

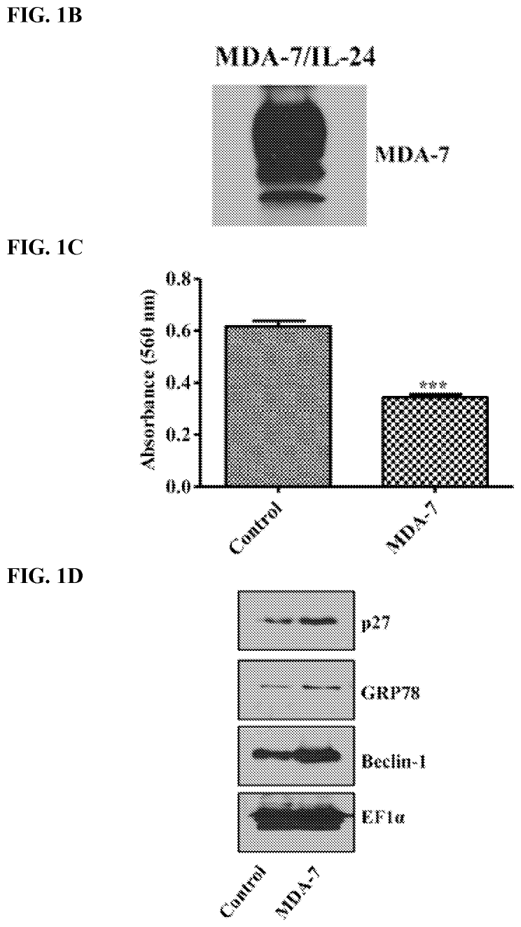

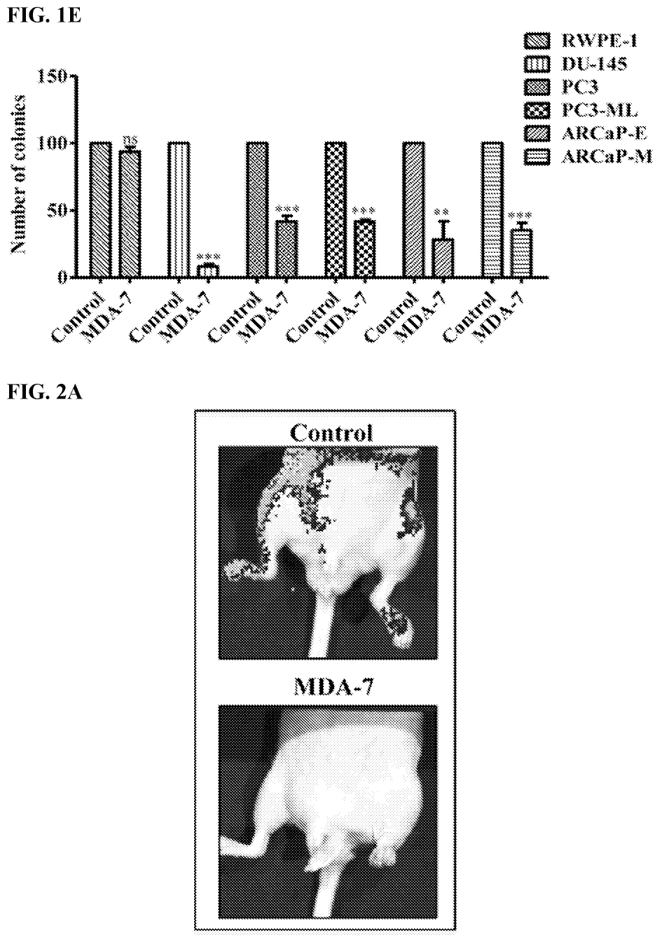

Recombinant MDA-7/IL-24 Selectively Inhibits PC Cell Growth: Recombinant MDA-7/IL-24 was purified using a His-based protein purification system as described above, and illustrated in (FIG. 1A). After purification, the quality of the purified protein was confirmed using anti-MDA-7 antibody by western blotting (FIG. 1B). To confirm biological activity, PC3-ML cells were treated with MDA-7/IL-24 protein, and anti-proliferative and potential molecular changes were evaluated by MTT assays and western blotting analyses, respectively (FIGS. 1C and 1D). Cell proliferation was significantly impaired following MDA-7/IL-24 treatment (FIG. 1C). Significant increases were observed in the levels of p27, GRP78, and Beclin-1 (FIG. 1D), which is consistent with studies where mda-7/IL-24 was delivered using an Adenovirus (25). Next, to evaluate the long-term effect of MDA-7/IL-24 on cell proliferation, colony formation (clonal) assays were performed in an assortment of PC cell lines. Results shown in FIG. 1E illustrate that MDA-7/IL-24 protein significantly reduced the proliferation of PC cells without affecting the proliferation capacity of immortalized normal primary human prostate epithelial cells (RWPE-1).

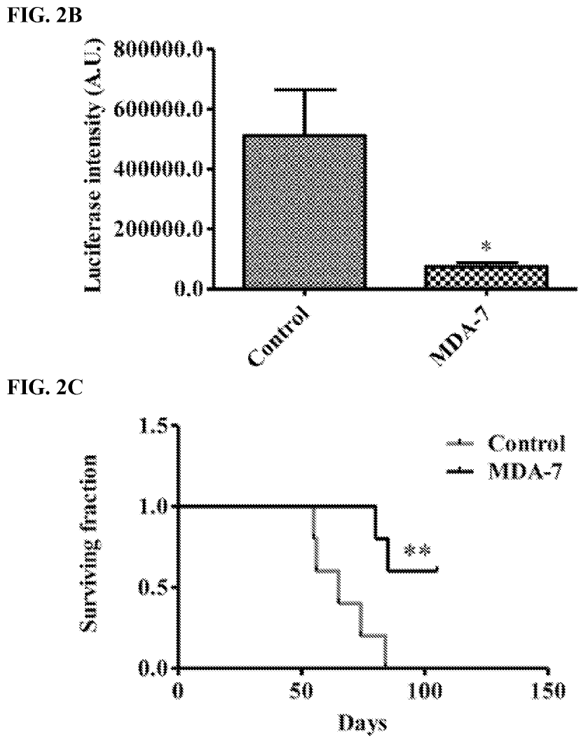

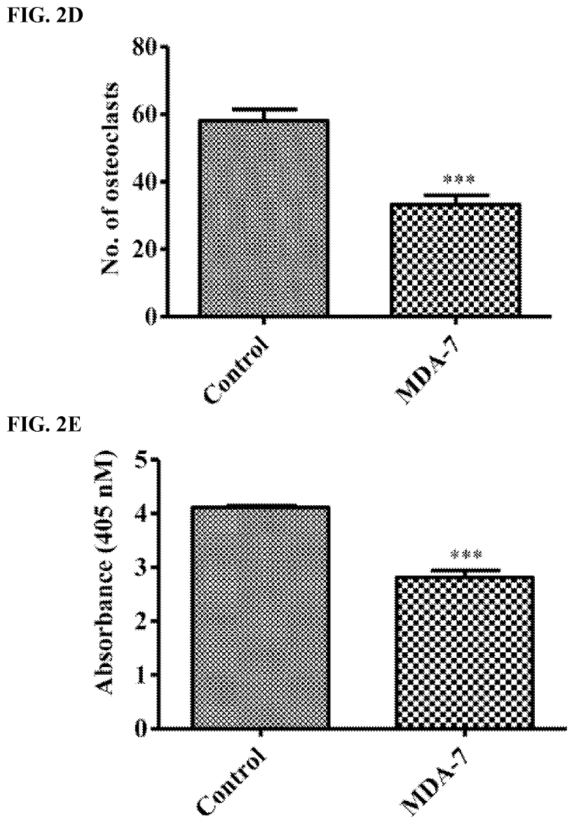

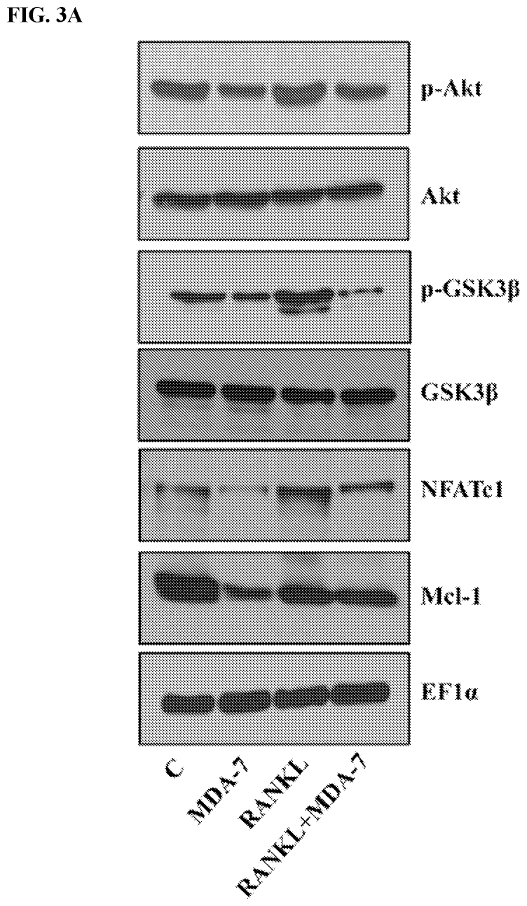

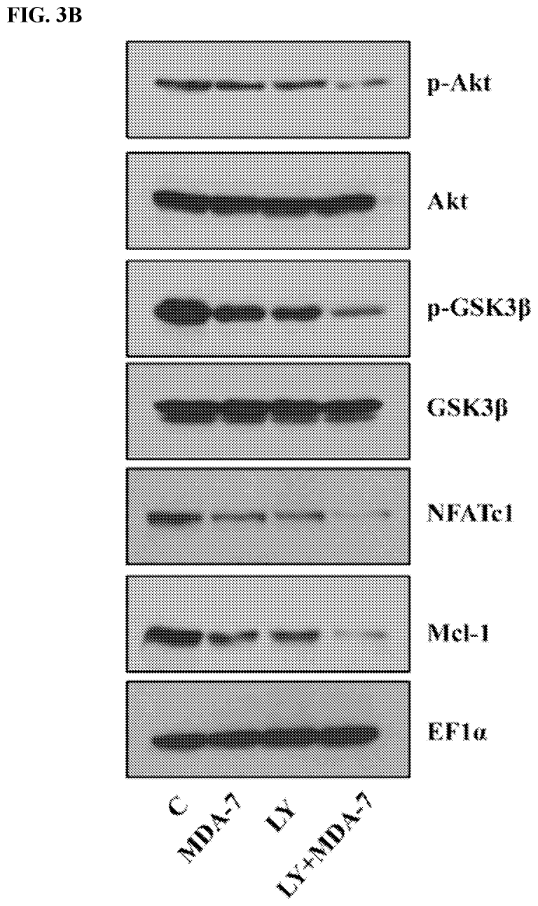

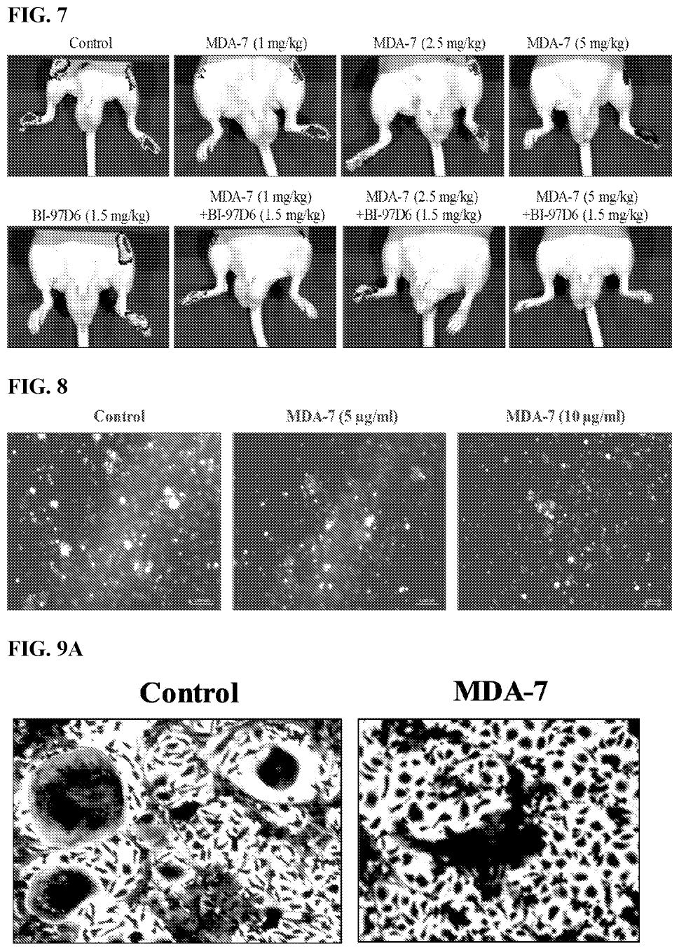

Recombinant MDA-7/IL-24 Decreases In Vivo PC Bone Metastasis: Use of recombinant MDA-7/IL-24 protein to suppress PC bone metastasis was tested using an experimental metastasis model. In this model, stable luciferase expressing PC3-ML cells were injected in male athymic nude mice through an intracardiac route to produce bone metastases. The expansion, invasion, and migration of PC3-ML cells were monitored by IVIS® imaging (15, 20, 23). Based on preliminary tests to optimize duration of treatment, optimum dose of MDA-7/IL-24, and the number of injections (illustrated in FIG. 7), animals were treated with MDA-7/IL-24 protein for three weeks with a total of 6 doses at 5 mg/kg through tail vein injection. BLI imaging followed development of metastatic lesions in bone. Robust bone metastasis was observed in the untreated control group, while there was significantly less evidence of metastasis in the MDA-7/IL-24-treated animals, as indicated by decreased BLI signals using IVIS® imaging (FIG. 2A). The luciferase intensities in the different groups of animals are shown in FIG. 2B. These results in a model of PC-induced bone metastasis, together with survival, which increased in MDA-7/IL-24-treated animals (FIG. 2C), support a therapeutic role of recombinant MDA-7/IL-24 protein in suppressing bone metastasis. To determine the effect of MDA-7/IL-24 on primary bone marrow in animals, cells from the bone cavity were isolated and treated with His-MDA-7 at different doses in vitro. No apparent toxicity was observed in primary bone marrow cells-treated with His-MDA-7 (FIG. 8).

MDA-7/IL-24 Inhibits RANKL-Induced Osteoclast Differentiation: To determine the osteoclastic activity in tumor bearing animals, either treated or un-treated with therapeutic, bone marrow cells were isolated from the femur and osteoclast differentiation was experimentally induced. MDA-7/IL-24-treated animals had significantly fewer osteoclasts as compared to the control group. This was quantified by counting the number of osteoclasts (FIG. 2D) and also measuring TRACP osteoclastic enzymatic activity (FIG. 2E). These initial results indicated a potential function of MDA-7/IL-24 in regulating osteoclast differentiation.

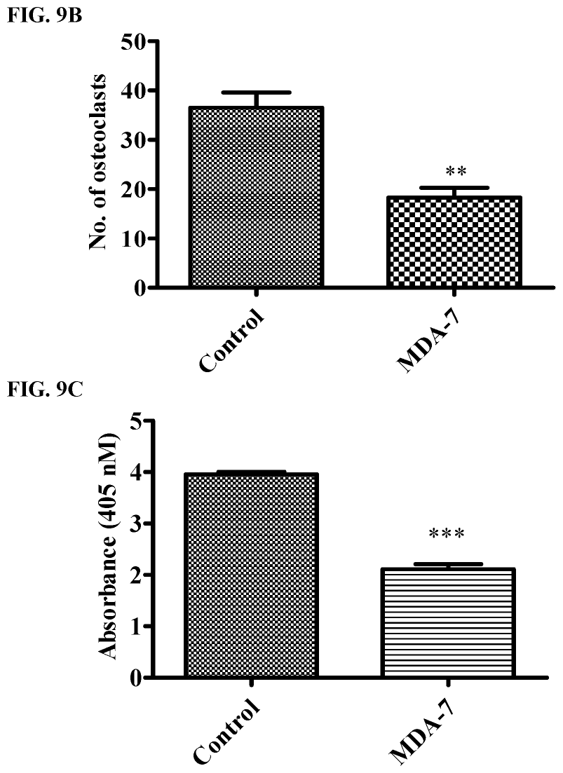

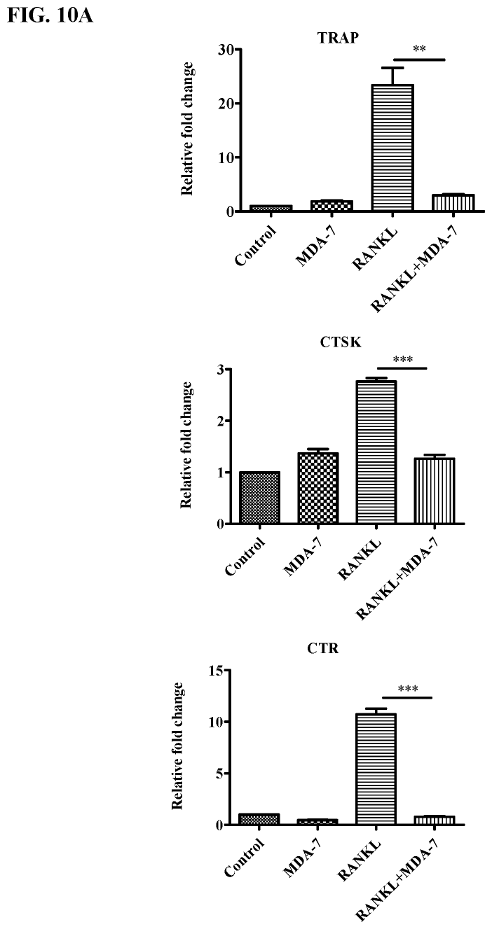

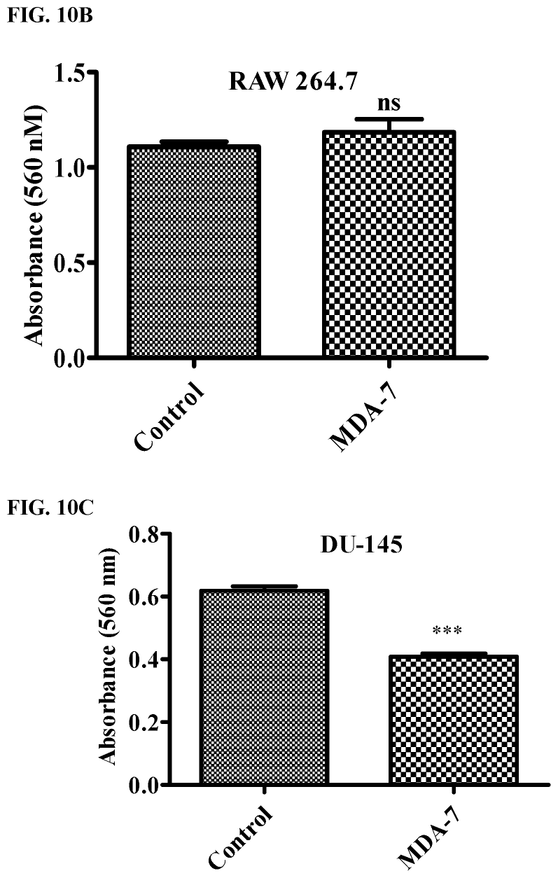

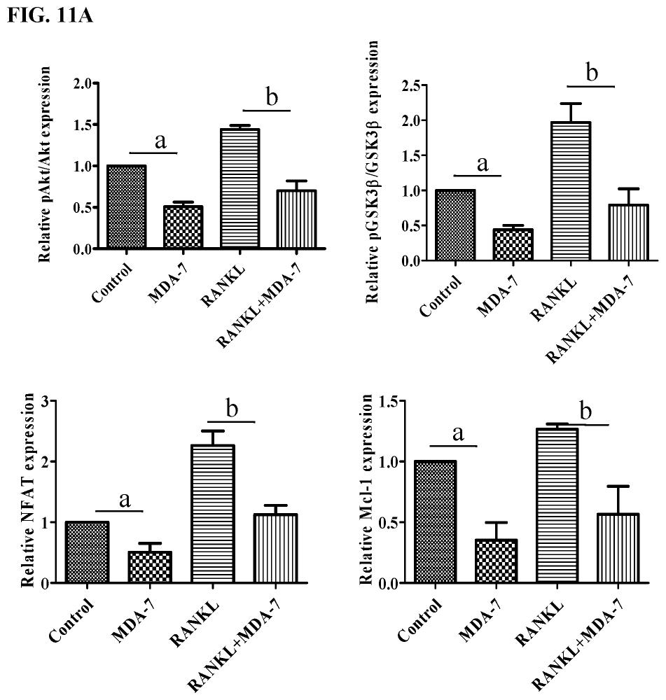

To investigate the effect of MDA-7/IL-24 on osteoclast differentiation, bone marrow cells from athymic nude mice were isolated and induced to differentiate with RANKL in the presence or absence of MDA-7/IL-24 protein. Five days after induction with RANKL, osteoclast differentiation was measured by counting multinucleated cells. Cells positively stained for TRAP and multinucleated cells were quantified under a light microscope (FIG. 9A). The number of osteoclasts and its activity was significantly reduced in the MDA-7/IL-24-treated group in comparison with controls (FIGS. 9B-C). To provide molecular insights into this inhibitory effect, we used a mouse macrophage cell line RAW 264.7, which can be induced to differentiate into osteoclasts when incubated with RANKL (32). Using RAW 264.7 cells, different genetic markers associated with osteoclastic differentiation were monitored (33). RAW 264.7 cells were treated with RANKL or MDA-7/IL-24 alone and in combination. RNA was isolated after 5 days of treatment and real time PCR quantified expression of TRAP, Cathepsin K (CTSK), and Calcitonin Receptor (CTR) genes. RANKL induced the expression of TRAP, CTSK and CTR gene expression. Addition of MDA-7/IL-24 protein attenuated RANKL-induced regulation of these genes (FIG. 10A). These data validate the hypothesis that MDA-7/IL-24 can mediate inhibition in RANKL-induced osteoclastic differentiation. MTT assays were performed to determine if MDA-7/IL-24 caused any growth suppression in this cell line. Proliferation of RAW 264.7 was not affected by MDA-7/IL-24, further illustrating the tumor-specificity of this cytokine (FIG. 10B). DU-145 (PC) cells were used as a positive control (FIG. 10C).

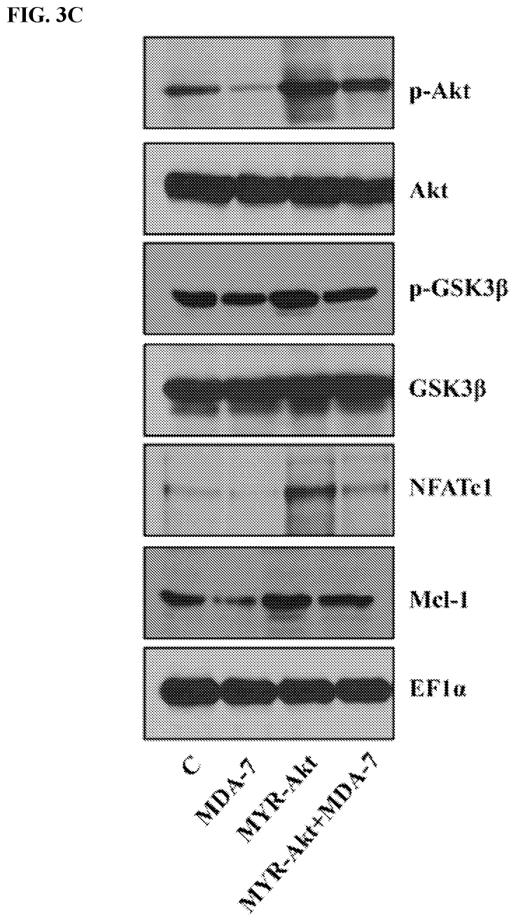

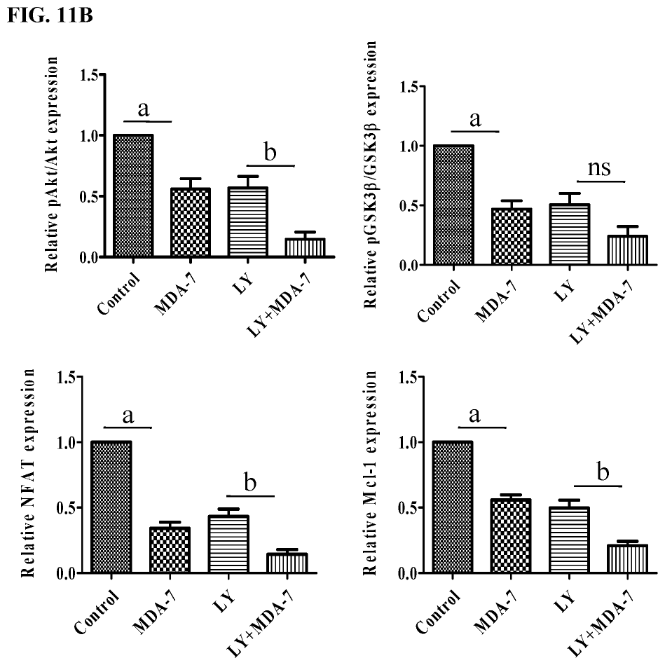

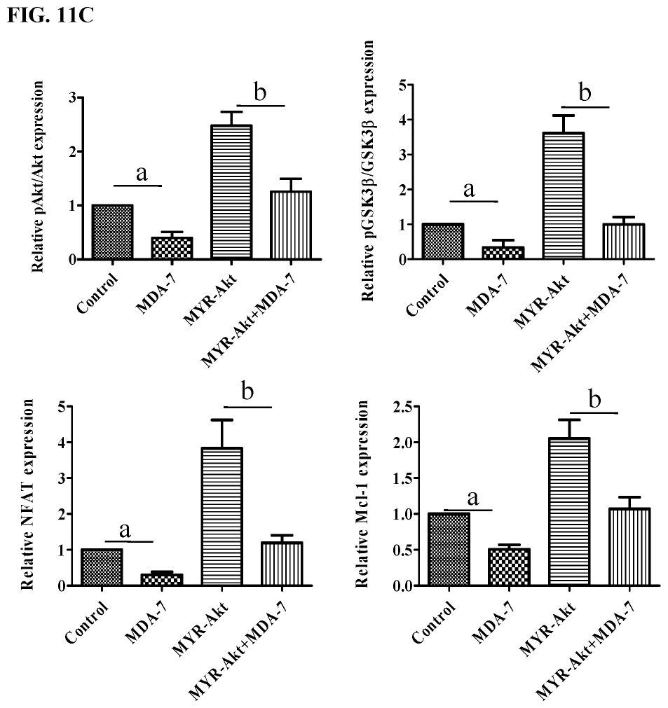

MDA-7/IL-24 Regulates AKT Signaling in Mouse Macrophage cells: To determine whether MDA-7/IL-24 protein exerts any effect on Akt activation in our model system, RAW 264.7 cells were treated with MDA-7/IL-24 protein in the presence or absence of RANKL. It was observed that RANKL induces Akt activation in RAW 264.7 cells, and this activated Akt was suppressed by MDA-7/IL-24 protein (FIGS. 3A and 11A). This data suggests a likely role of Akt inhibition in MDA-7/IL-24-mediated down regulation of osteoclast differentiation. The effect of Akt inhibition by a PI3K kinase inhibitor (LY294002) was also studied in the signaling cascade involving NFAT, Mcl-1, and Akt (FIGS. 3B and 11B). This data illustrates the cellular signaling of Akt pathway-related genes mediated by RANKL and MDA-7/IL-24.

A constitutively active form of Akt (MYR-Akt) was also tested. Cells were transfected with MYR-Akt and treated with MDA-7/IL-24. As shown in FIGS. 3C and 11C, treatment with MDA-7/IL-24 profoundly inhibited Mcl-1 expression, which was rescued by overexpression of a constitutively active Akt (MYR-Akt). These results illustrate the role of Akt in MDA-7/IL-24-mediated suppression in osteoclastic differentiation.

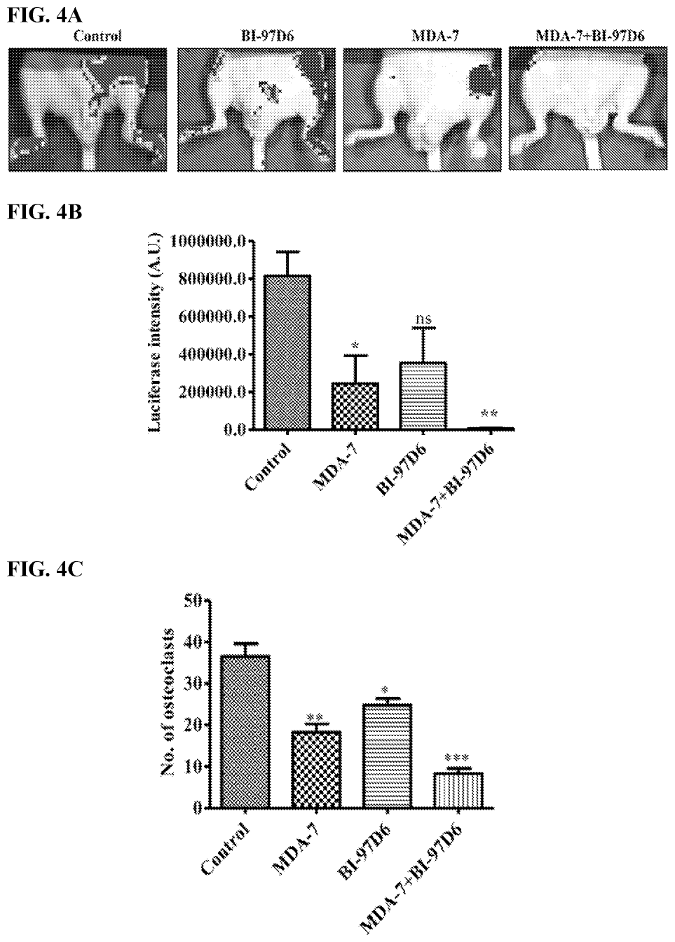

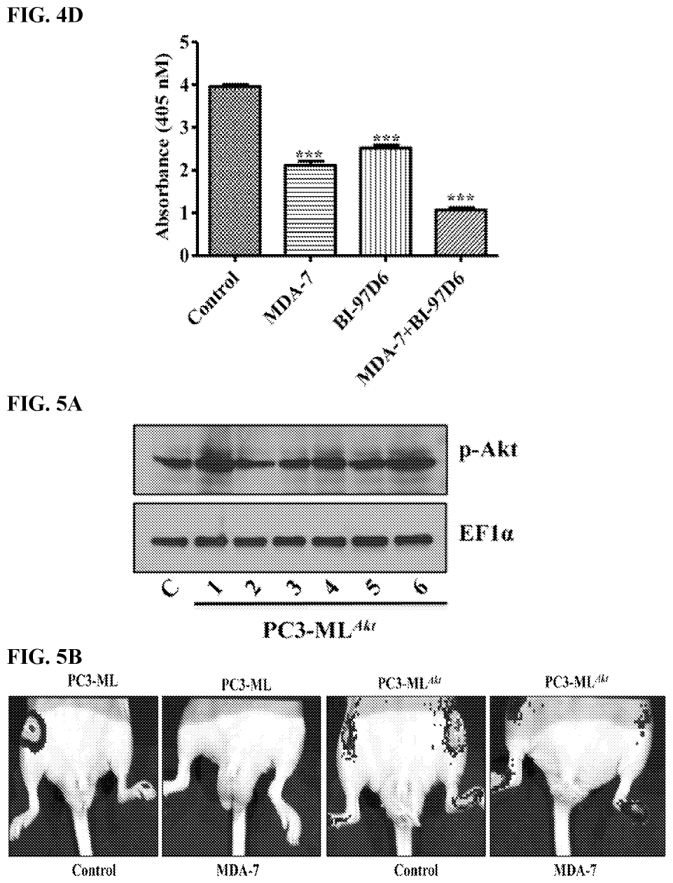

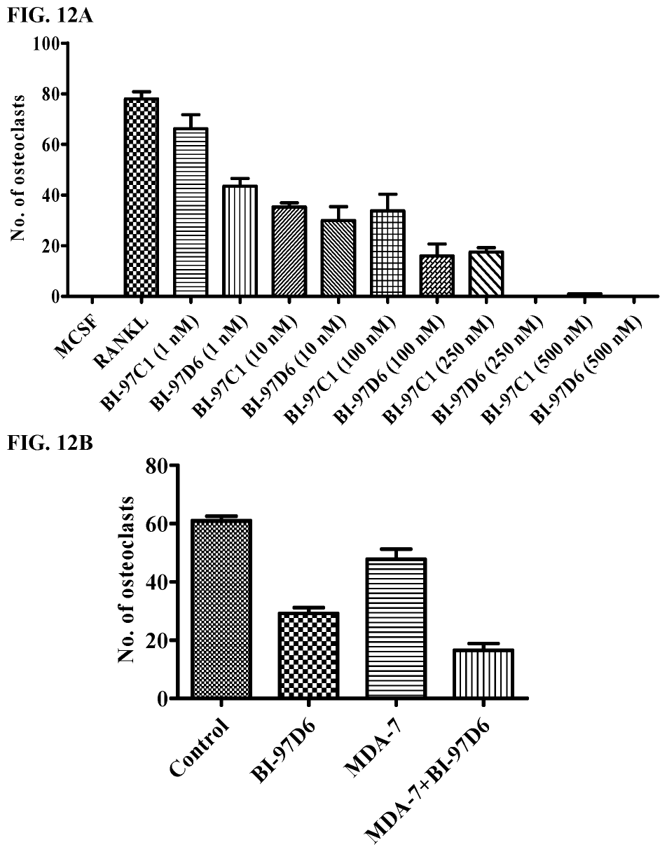

Mcl-1 Inhibitor, BI-97D6, Synergizes with MDA-7/IL-24 in Suppressing PC Bone Metastasis: The effect of MDA-7/IL-24 protein in combination with BI-97D6, a small molecule Mcl-1 inhibitor, was tested in male athymic nude mice injected with PC3-ML cells by the intracardiac route, as a model for bone metastases. Animals were treated through tail vein injection with MDA-7/IL-24 protein for three weeks with a total of 6 doses at 5 mg/kg. BI-97D6 was administered through intraperitoneal route at 1.5 mg/kg body weight with a total of 6 doses (FIG. 4A). A significant level of bone metastasis was evident in the control group, while MDA-7/IL-24 protein treatment resulted in significantly fewer metastatic lesions (FIG. 4A). Treatment with BI-97D6 alone also showed some inhibitory effects on bone metastasis development; however, when combined with MDA-7/IL-24 a dramatic inhibition in bone metastasis development was seen, indicating a combinatorial therapeutic role of MDA-7/IL-24 protein with an Mcl-1 inhibitor in bone metastasis (FIG. 4A). The luciferase intensities are illustrated in FIG. 4B. This combination also reduced osteoclast differentiation. Dose response assays showed a down regulation in the number of osteoclasts when primary bone marrow cells were induced with MCSF and RANKL and treated with Mcl-1 inhibitors (FIG. 12A). Synergy in the inhibition of osteoclast differentiation was also evident following treatment with the combination of MDA-7/IL-24 and BI-97D6 (FIG. 12B).

Suppression of osteoclast differentiation was also evaluated using an in vivo metastatic model (FIG. 4C). Bone marrow cells were isolated after sacrifice of mice and osteoclast differentiation was induced. The MDA-7/IL-24- or BI-97D6-treated groups of animals had significantly fewer osteoclasts as compared to control groups. The bone marrow cells isolated from the MDA-7/IL-24 and BI-97D6 animals formed statistically fewer osteoclasts following induction with RANKL. This was evident by counting osteoclasts (FIG. 4C) and also by measuring TRACP osteoclastic enzymatic activity (FIG. 4D).

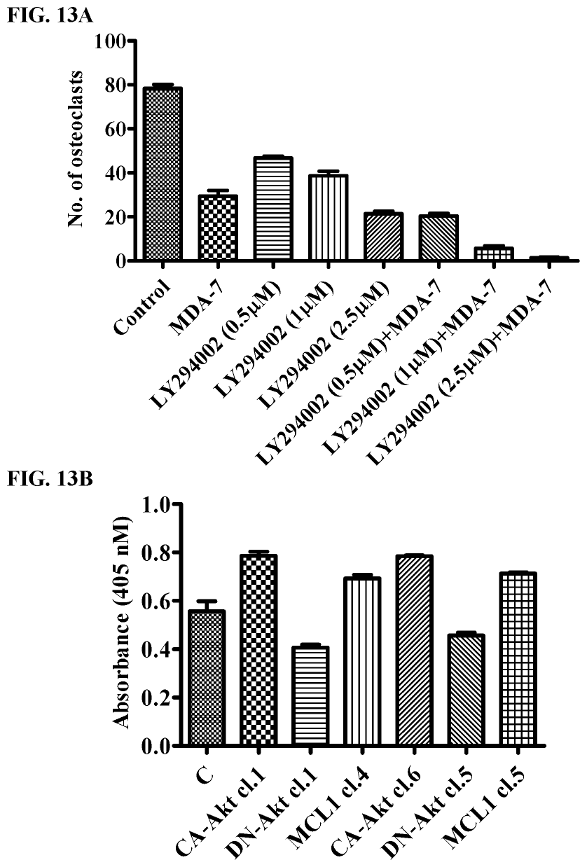

Akt Regulates Bone metastasis of PC Cells In Vivo. To evaluate a role of Akt in PC bone metastasis, the activity of Akt was inhibited using the PI3 kinase inhibitor LY294002 (38) and effects on osteoclast differentiation were determined. The PI3 kinase inhibitor LY294002 synergized with MDA-7/IL-24 protein to reduce osteoclast differentiation (FIG. 13A). Additionally, treatment with LY294002 in combination with MDA-7/IL-24 protein resulted in decreased expression of downstream molecules including NFAT and Mcl-1 (FIGS. 3B and 11B). As a further test, RAW 264.7 cells were stably transfected with CA-Akt, DN-Akt, and Mcl-1, and osteoclast differentiation was determined. Akt and Mcl-1 overexpressing clones formed more osteoclasts as compared to controls, whereas DN-Akt overexpressed RAW 264.7 cells formed fewer osteoclasts (FIG. 13B). Taken together, these results indicate synergistic effects for combinations of MDA-7/IL-24 protein with a PI3K inhibitor or an Akt inhibitor.

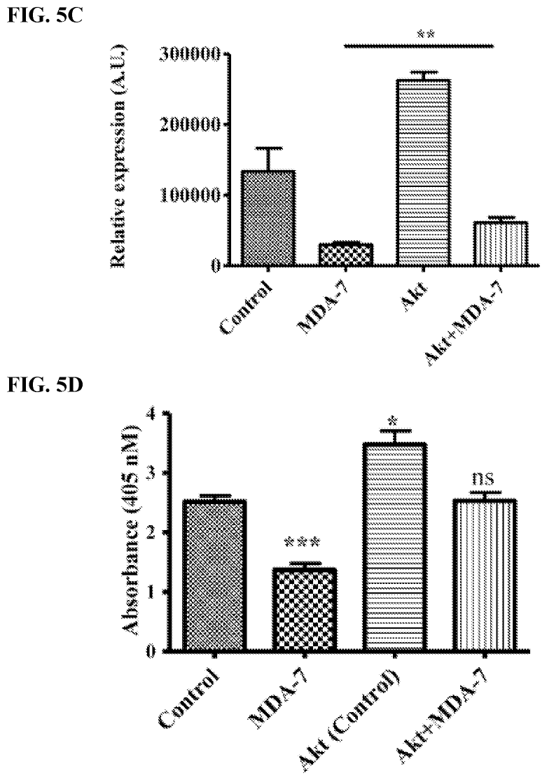

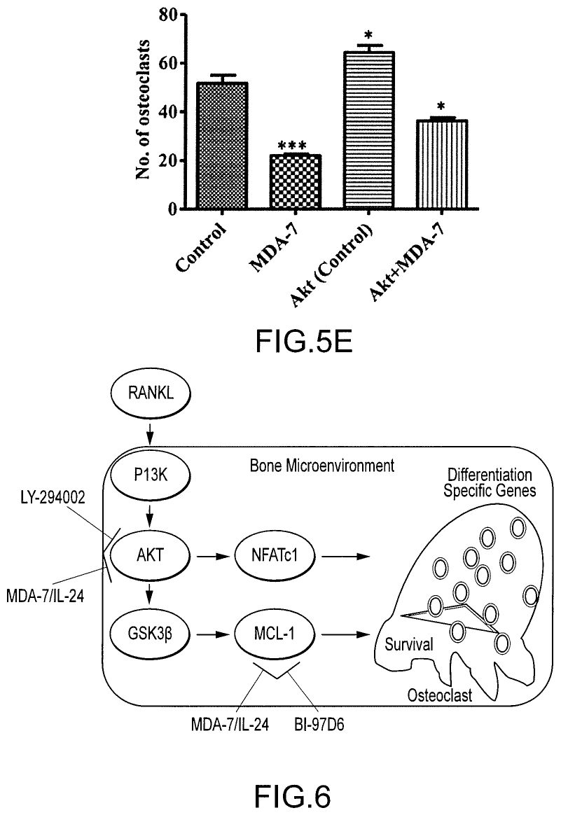

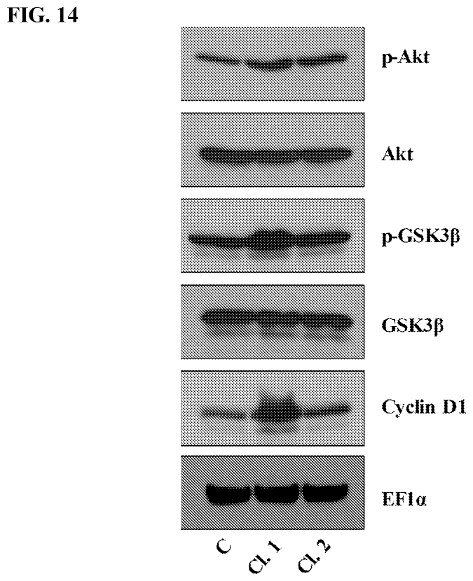

To evaluate the role of Akt in vivo, metastatic PC3-ML cells and PC3-ML cells over expressing constitutively active Akt (PC3-MI/**) (FIG. 5A) carrying firefly luciferase were injected into male athymic nude mice through the intracardiac route. Clone 1 was used for the in vivo studies, which was validated for expression of Akt downstream pathway gene expression by western blotting (FIG. 14). Proliferation, invasion, and migration of the tumor cells were monitored by IVIS® imaging. Treatment with MDA-7/IL-24 protein was continued for three weeks with a total of 6 doses at 5 mg/kg through the tail vein. Significant bone metastasis was apparent in the control group, while there was a significant decrease in metastasis in MDA-7/IL-24-treated animals. The Akt overexpressing PC3-ML cells were more metastatic than the control PC3-ML cells. Treatment with MDA-7/IL-24 inhibited metastasis by the PC3-ML^{Akt} group; however, this effect was reduced in comparison with His-MDA-7-treated parental PC3-ML cells. These observations illustrate the significance of Akt in PC-mediated bone metastasis development, which can be partially abrogated by MDA-7/IL-24-treatment (FIG. 5B). These data were further substantiated by quantification of luciferase intensities in the different experimental animal groups (FIG. 5C). To investigate osteoclastic differentiation in these animals, bone marrow cells were isolated after completion of the study. Osteoclast differentiation was induced as described above. Bone marrow isolated from MDA-7/IL-24-treated animals showed less osteoclastic activity as compared to the control group. Bone marrow from animals injected with elevated Akt-stable expression cells showed more osteoclastic activity, which decreased following treatment with MDA-7/IL-24 protein. Osteoclast differentiation assays further illustrated the importance of Akt in PC-mediated bone metastasis (FIGS. 5D and 5E). A schematic representation of the proposed role of MDA-7/IL-24 protein in PC-mediated bone metastasis through modulation of the bone microenvironment is presented in FIG. 6. Additional effects of MDA-7/IL-24 that may contribute to inhibition of PC-induced bone metastasis include direct killing of PC cells (through apoptosis or toxic autophagy), inhibition of angiogenesis and immune-mediated anti-PC activity (29, 30).

These results illustrate that purified MDA-7/IL-24 recombinant protein inhibits the metastasis of PC cells to bone. Treatment with recombinant MDA-7/IL-24 significantly reduced the occurrence of bone metastasis in an experimental in vivo model and the effect was more vigorous when combined with an Mcl-1 inhibitor. In vitro studies suggest that MDA-7/IL-24 reduced osteoclast differentiation induced by RANKL, partly through inhibition of phosphorylated-Akt and Mcl-1. Accordingly, MDA-7/IL-24 protein and an Mcl-1-targeted small molecule inhibitor hold potential as efficacious therapeutics against PC bone metastasis. Moreover, the results described above indicate that recombinant MDA-7/IL-24 can be delivered repeatedly systemically in mice without promoting toxicity and can inhibit the development of PC bone metastasis.

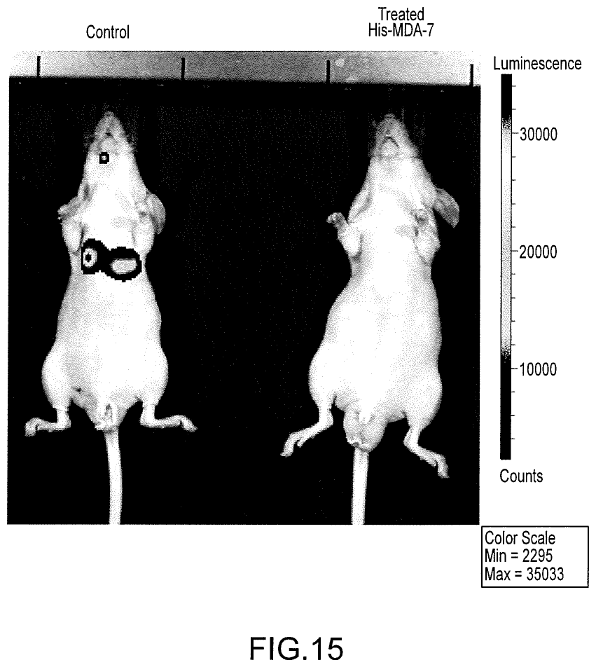

The effect of His-tagged MDA-7/IL-24 on metastasis was also observed to be more global, since treatment of animals receiving intracardiac delivery of PC3-ML cells also suppressed development of lung metastases (FIG. 15). Also, considering effects of MDA-7/IL-24 protein on bone, it is further expected that metastases from other cancer types to bone (e.g., lung cancer, and breast cancer) would likewise be prevented/treated.

### REFERENCES

1. Rigaud J, Tiguert R, Le Normand L, Karam G, Glemain P, Buzelin J M, el al. Prognostic value of bone scan in patients with metastatic prostate cancer treated initially with androgen deprivation therapy. J Urol 2002; 168:1423-6.
2. Ye L, Kynaston H G, Jiang W G. Bone metastasis in prostate cancer: molecular and cellular mechanisms (Review). Int J Mol Med 2007; 20:103-11.
3. Coghlin C, Murray G I. Current and emerging concepts in tumour metastasis. J Pathol 2010; 222:1-15.
4. Tanaka Y, Nakayamada S, Okada Y. Osteoblasts and osteoclasts in bone remodeling and inflammation. Current drug targets Inflammation and allergy 2005; 4:325-8.
5. Quinn B A, Dash R, Azab B, Sarkar S, Das S K, Kumar S, et al. Targeting Mcl-1 for the therapy of cancer. Expert opinion on investigational drugs 2011; 20:1397-411.
6. Beroukhim R, Mermel C H, Porter D, Wei G, Raychaudhuri S, Donovan J, et al. The landscape of somatic copy-number alteration across human cancers. Nature 2010; 463:899-905.
7. Sutherland K A, Rogers H L, Tosh D, Rogers M J. RANKL increases the level of Mcl-1 in osteoclasts and reduces bisphosphonate-induced osteoclast apoptosis in vitro. Arthritis Res Ther 2009; 11:R58.
8. Masuda H, Hirose J, Omata Y, Tokuyama N, Yasui T, Kadono Y, et al. Anti-apoptotic Bcl-2 family member Mcl-1 regulates cell viability and bone-resorbing activity of osteoclasts. Bone 2014; 58:1-10.
9. Wei J, Kitada S, Rega M F, Stebbins J L, Zhai D, Cellitti J, et al. Apogossypol derivatives as pan-active inhibitors of antiapoptotic B-cell lymphoma/leukemia-2 (Bcl-2) family proteins. J Med Chem 2009; 52:4511-23.
10. Wei J, Kitada S, Rega M F, Emdadi A, Yuan H, Cellitti J, el al. Apogossypol derivatives as antagonists of antiapoptotic Bcl-2 family proteins. Mol Cancer Ther 2009; 8:904-13.
11. Wei J, Rega M F, Kitada S, Yuan H, Zhai D, Risbood P, et al. Synthesis and evaluation of Apogossypol atropisomers as potential Bcl-xL antagonists. Cancer Lett 2009; 273:107-13.
12. Kitada S, Kress C L, Krajewska M, Jia L, Pellecchia M, Reed J C. Bcl-2 antagonist apogossypol (NSC736630) displays single-agent activity in Bcl-2-transgenic mice and has superior efficacy with less toxicity compared with gossypol (NSC19048). Blood 2008; 111:3211-9.
13. Jia L, Coward L C, Kerstner-Wood C D, Cork R L, Gorman G S, Noker P E, et al. Comparison of pharmacokinetic and metabolic profiling among gossypol, apogossypol and apogossypol hexaacetate. Cancer Chemother Pharmacol 2008; 61:63-73.
14. Wei J, Stebbins J L, Kitada S, Dash R, Placzek W, Rega M F, et al. BI-97C1, an optically pure Apogossypol derivative as pan-active inhibitor of antiapoptotic B-cell lymphoma/leukemia-2 (Bcl-2) family proteins. J Med Chem 2010; 53:4166-76.
15. Azab B, Dash R, Das S K, Bhutia S K, Shen X N, Quinn B A, et al Enhanced delivery of mda-7/IL-24 using a serotype chimeric adenovirus (Ad.5/3) in combination with the Apogossypol derivative BI-97C1 (Sabutoclax) improves therapeutic efficacy in low CAR colorectal cancer cells. Journal of cellular physiology 2012; 227:2145-53.
16. Dash R, Azab B, Shen X N, Sokhi U K, Sarkar S, Su Z Z, et al. Developing an effective gene therapy for prostate cancer: New technologies with potential to translate from the laboratory into the clinic. Discov Med 2011; 11:46-56.
17. Dash R, Azab B, Quinn B A, Shen X, Wang X Y, Das S K, el al. Apogossypol derivative BI-97C1 (Sabutoclax) targeting Mcl-1 sensitizes prostate cancer cells to mda-7/IL-24-mediated toxicity. Proceedings of the National Academy of Sciences of the United States of America 2011; 108:8785-90.
18. Dash R, Dmitriev I, Su Z Z, Bhutia S K, Azab B, Vozhilla N, et al. Enhanced delivery of mda-7/IL-24 using a serotype chimeric adenovirus (Ad.5/3) improves therapeutic efficacy in low CAR prostate cancer cells. Cancer Gene Ther 2010; 17:447-56.
19. Das S K, Sarkar S, Dash R, Dent P, Wang X Y, Sarkar D, et al. Chapter One-Cancer terminator viruses and approaches for enhancing therapeutic outcomes. Adv Cancer Res 2012; 115:1-38.
20. Bhatnagar A, Wang Y, Mease R C, Gabrielson M, Sysa P, Minn I, et al. AEG-1 promoter-mediated imaging of prostate cancer. Cancer Res 2014; 74:5772-81.
21. Su Z, Emdad L, Sauane M, Lebedeva I V, Sarkar D, Gupta P, et al. Unique aspects of mda-7/IL-24 antitumor bystander activity: establishing a role for secretion of MDA-7/IL-24 protein by normal cells. Oncogene 2005; 24:7552-66.
22. Wei J, Stebbins J L, Kitada S, Dash R, Zhai D, Placzek W J, et al. An optically pure apogossypolone derivative as potent pan-active inhibitor of anti-apoptotic bcl-2 family proteins. Front Oncol 2011; 1:28.
23. Sarkar S, Quinn B A, Shen X N, Dash R, Das S K, Emdad L, et al. Therapy of prostate cancer using a novel cancer terminator virus and a small molecule BH-3 mimetic. Oncotarget 2015; 6:10712-27.
24. Dash R, Bhoopathi P, Das S K, Sarkar S, Emdad L, Dasgupta S, et al. Novel mechanism of MDA-7/IL-24 cancer-specific apoptosis through SARI induction. Cancer Res 2014; 74:563-74.
25. Pradhan A K, Talukdar S, Bhoopathi P, Shen X N, Emdad L, Das S K et al. mda-7/IL-24 Mediates Cancer Cell-Specific Death via Regulation of miR-221 and the Beclin-1 Axis. Cancer Res 2017; 77:949-59.
26. Dash R, Richards J E, Su Z Z, Bhutia S K, Azab B, Rahmani M, et al. Mechanism by which Mcl-1 regulates cancer-specific apoptosis triggered by mda-7/IL-24, an IL-10-related cytokine. Cancer Res 2010; 70:5034-45.
27. Pradhan A K, Mohapatra A D, Nayak K B, Chakraborty S. Acetylation of the proto-oncogene EVI1 abrogates Bcl-xL promoter binding and induces apoptosis. PloS one 2011; 6:e25370.
28. Bradley E W, Oursler M J. Osteoclast culture and resorption assays. Methods in molecular biology 2008; 455:19-35.
29. Menezes M E, Bhatia S, Bhoopathi P, Das S K, Emdad L, Dasgupta S, et al. MDA-7/IL-24: multifunctional cancer killing cytokine. Adv Exp Med Biol 2014; 818:127-53.
30. Fisher P B. Is mda-7/IL-24 a “magic bullet” for cancer? Cancer Res 2005; 65:10128-38.
31. Yin J J, Pollock C B, Kelly K. Mechanisms of cancer metastasis to the bone. Cell Res 2005; 15:57-62.
32. Collin-Osdoby P, Osdoby P. RANKL-mediated osteoclast formation from murine RAW 264.7 cells. Methods in molecular biology 2012; 816:187-202.
33. Takeshita S, Kaji K, Kudo A. Identification and characterization of the new osteoclast progenitor with macrophage phenotypes being able to differentiate into mature osteoclasts. J Bone Miner Res 2000; 15:1477-88.
34. Moon J B, Kim J H, Kim K, Youn B U, Ko A, Lee S Y, el al. Akt induces osteoclast differentiation through regulating the GSK3beta/NFATc1 signaling cascade. J Immunol 2012; 188:163-9.
35. Liou S F, Hsu J H, Lin I L, Ho M L, Hsu P C, Chen L W, et al. KMUP-1 suppresses RANKL-induced osteoclastogenesis and prevents ovariectomy-induced bone loss: roles of MAPKs, Akt, NF-kappaB and calcium/calcineurin/NFATc1 pathways. PloS one 2013; 8:e69468.
36. Inoue S, Branch C D, Gallick G E, Chada S, Ramesh R. Inhibition of Src kinase activity by Ad-mda7 suppresses vascular endothelial growth factor expression in prostate carcinoma cells. Mol Ther 2005; 12:707-15.
37. Valero V, 3rd, Wingate H, Chada S, Liu Y, Palalon F, Mills G, et al. MDA-7 results in downregulation of AKT concomitant with apoptosis and cell cycle arrest in breast cancer cells. Cancer Gene Ther 2011; 18:510-9.
38. Vlahos C J, Matter W F, Hui K Y, Brown R F. A specific inhibitor of phosphatidylinositol 3-kinase, 2-(4-morpholinyl)-8-phenyl-4H-l-benzopyran-4-one (LY294002). J Biol Chem 1994; 269:5241-8.
39. Riihimaki M, Thomsen H, Brandt A, Sundquist J, Hemminki K. What do prostate cancer patients die of? Oncologist 2011; 16:175-81.
40. Cunningham C C, Chada S, Merritt J A, Tong A, Senzer N, Zhang Y, et al. Clinical and local biological effects of an intratumoral injection of mda-7 (IL24; INGN 241) in patients with advanced carcinoma: a phase I study. Mol Ther 2005; 11:149-59.
41. Nishikawa T, Ramesh R, Munshi A, Chada S, Meyn R E. Adenovirus-mediated mda-7 (IL24) gene therapy suppresses angiogenesis and sensitizes NSCLC xenograft tumors to radiation. Mol Ther 2004; 9:818-28.
42. Ramesh R, Mhashilkar A M, Tanaka F, Saito Y, Branch C D, Sieger K et al. Melanoma differentiation-associated gene 7/interleukin (IL)-24 is a novel ligand that regulates angiogenesis via the IL-22 receptor. Cancer Res 2003; 63:5105-13.
43. Bhutia S K, Dash R, Das S K, Azab B, Su Z Z, Lee S G, et al. Mechanism of autophagy to apoptosis switch triggered in prostate cancer cells by antitumor cytokine melanoma differentiation-associated gene 7/interleukin-24. Cancer Res 2010; 70:3667-76.
44. Ramesh R, I to I, Gopalan B, Saito Y, Mhashilkar A M, Chada S. Ectopic production of MDA-7/IL-24 inhibits invasion and migration of human lung cancer cells. Mol Ther 2004; 9:510-8.
45. Dent P, Yacoub A, Grant S, Curiel D T, Fisher P B. MDA-7/IL-24 regulates proliferation, invasion and tumor cell radiosensitivity: a new cancer therapy? J Cell Biochem 2005; 95:712-9.
46. Sauane M, Su Z Z, Gupta P, Lebedeva I V, Dent P, Sarkar D, el al. Autocrine regulation of mda-7/IL-24 mediates cancer-specific apoptosis. Proceedings of the National Academy of Sciences of the United States of America 2008; 105:9763-8.
47. Sauane M, Gopalkrishnan R V, Choo H T, Gupta P, Lebedeva I V, Yacoub A, et al. Mechanistic aspects of mda-7/IL-24 cancer cell selectivity analysed via a bacterial fusion protein. Oncogene 2004; 23:7679-90.
48. Leverson J D, Zhang H, Chen J, Tahir S K, Phillips D C, Xue J, et al. Potent and selective small-molecule MCL-1 inhibitors demonstrate on-target cancer cell killing activity as single agents and in combination with ABT-263 (navitoclax). Cell death & disease 2015; 6:e1590.
49. Minagawa N, Kruglov E A, Dranoff J A, Robert M E, Gores G J, Nathanson M H. The anti-apoptotic protein Mcl-1 inhibits mitochondrial Ca2+ signals. J Biol Chem 2005; 280:33637-44.
50. Bhoopathi P, Lee N, Pradhan A K, Shen X N, Das S K, Sarkar D, et al-mda-7/IL-24 Induces Cell Death in Neuroblastoma through a Novel Mechanism Involving AIF and ATM. Cancer Res 2016; 76:3572-82.

### EMBODIMENTS

Embodiment 1. A method of preventing metastasis to bone in a subject with cancer, the method comprising administering an effective amount of a composition comprising an MDA-7/IL-24 protein to the subject.

Embodiment 2. A method of treating bone metastasis in a subject with cancer, the method comprising administering an effective amount of a composition comprising an MDA-7/IL-24 protein to the subject.

Embodiment 3. The method of embodiment 1 or 2, wherein the MDA-7/IL-24 protein is a purified protein.

Embodiment 4. The method of any one of embodiments 1-3, wherein the MDA-7/IL-24 protein is a mature protein.

Embodiment 5. The method of any one of embodiments 1-4, wherein the administering comprises administering to a bone of said subject.

Embodiment 6. The method of any one of embodiments 1-5, wherein the cancer is prostate cancer.

Embodiment 7. The method of embodiment 6, wherein the prostate cancer comprises cancer cells having an increased expression of one or more of Mcl-1, RANKL, Bcl-2, Bcl-xL, and Akt, relative to normal prostate cells.

Embodiment 8. The method of any one of embodiments 1-7, wherein the composition further comprises an Mcl-1 inhibitor.

Embodiment 9. The method of embodiment 8, wherein the Mcl-1 inhibitor is BI-97D6.

Embodiment 10. The method of any one of embodiments 1-9, wherein the composition further comprises a phosphoinositide 3-kinase (PI3K) inhibitor.

Embodiment 11. The method of embodiment 10, wherein the PI3K inhibitor is LY294002.

Embodiment 12. The method of any one of embodiments 1-11, wherein the effective amount is an amount that is substantially non-toxic to primary bone marrow cells or normal primary human prostate epithelial cells.

Embodiment 13. The method of any one of embodiments 1-12, wherein the effective amount is an amount that inhibits osteoclast differentiation.

Embodiment 14. The method of any one of embodiments 1-13, wherein the MDA-7/IL-24 protein comprises an amino acid sequence that is at least 90% identical to SEQ ID NO: 3.

Embodiment 15. The method of embodiment 14, wherein the MDA-7/IL-24 protein comprises an amino acid sequence that is at least 95% identical to SEQ ID NO: 3.

Embodiment 16. The method of embodiment 14 or 15, wherein the MDA-7/IL-24 protein is capable of activating an IL-20/IL-22 receptor complex of a cancer cell of the subject.

Embodiment 17. The method of embodiment 14, wherein the MDA-7/IL-24 protein comprises an amino acid sequence of SEQ ID NO: 3.

Embodiment 18. A composition comprising an MDA-7/IL-24 protein and one or both of an Mcl-1 inhibitor and a PI3K inhibitor.

Embodiment 19. The composition of embodiment 18, wherein the MDA-7/IL-24 protein is a purified protein.

Embodiment 20. The composition of embodiment 18 or 19, wherein the MDA-7/IL-24 protein is a mature protein.

Embodiment 21. The composition of any one of embodiments 18-20, wherein the composition comprises an Mcl-1 inhibitor.

Embodiment 22. The composition of embodiment 21, wherein the Mcl-1 inhibitor is BI-97D6.

Embodiment 23. The composition of any one of embodiments 18-22, wherein the composition comprises a PI3K inhibitor.

Embodiment 24. The composition of embodiment 23, wherein the PI3K inhibitor is LY294002.

Embodiment 25. The composition of any one of embodiments 18-24, wherein the MDA-7/IL-24 protein comprises an amino acid sequence that is at least 90% identical to SEQ ID NO: 3.

Embodiment 26. The composition of embodiment 25, wherein the MDA-7/IL-24 protein comprises an amino acid sequence that is at least 95% identical to SEQ ID NO: 3.

Embodiment 27. The composition of embodiment 25 or 26, wherein the MDA-7/IL-24 protein is capable of activating an IL-20/IL-22 receptor complex of a cancer cell.

Embodiment 28. The composition of embodiment 25, wherein the MDA-7/IL-24 protein comprises an amino acid sequence of SEQ ID NO: 3.

Embodiment 29. The composition of any one of embodiments 18-28, further comprising a pharmaceutically acceptable excipient.

Embodiment 30. The composition of any one of embodiments 18-29 for use in preventing or treating bone metastasis in a subject with cancer.

Embodiment 31. The composition of embodiment 26, wherein the cancer is prostate cancer.

Embodiment 32. The composition of embodiment 31, wherein the prostate cancer comprises cancer cells having an increased expression of one or more of Mcl-1, RANKL, Bcl-2, Bcl-xL, and Akt, relative to normal prostate cells.

Embodiment 33. Use of a composition according to any one of embodiments 18-29 in the manufacture of a medicament for the prevention or treatment of bone metastasis in a subject with cancer.

Embodiment 34. Use of a composition according to any one of embodiments 18-29 in the manufacture of a medicament for the prevention or treatment of bone metastasis according to the method of any one of embodiments 1-17.

Embodiment 35. A kit comprising the MDA-7/IL-24 protein and one or both of the Mcl-1 inhibitor and the PI3K inhibitor of any one of the compositions of embodiments 18-29.

What is claimed is:

1. A method of preventing metastasis to bone in a subject with cancer, the method comprising administering an effective amount of a composition comprising an MDA-7/IL-24 protein to the subject.

2. A method of treating bone metastasis in a subject with cancer, the method comprising administering an effective amount of a composition comprising an MDA-7/IL-24 protein to the subject.

3. The method of claim 1 or 2, wherein the MDA-7/IL-24 protein is a purified protein.

4. The method of any one of claims 1-3, wherein the MDA-7/IL-24 protein is a mature protein.

5. The method of any one of claims 1-4, wherein the administering comprises administering to a bone of said subject.

6. The method of any one of claims 1-5, wherein the cancer is prostate cancer.

7. The method of claim 6, wherein the prostate cancer comprises cancer cells having an increased expression of one or more of Mcl-1, RANKL, Bcl-2, Bcl-xL, and Akt, relative to normal prostate cells.

8. The method of any one of claims 1-7, wherein the composition further comprises an Mcl-1 inhibitor.

9. The method of claim 8, wherein the Mcl-1 inhibitor is BI-97D6.

10. The method of any one of claims 1-9, wherein the composition further comprises a phosphoinositide 3-kinase (PI3K) inhibitor.

11. The method of claim 10, wherein the PI3K inhibitor is LY294002.

12. The method of any one of claims 1-11, wherein the effective amount is an amount that is substantially non-toxic to primary bone marrow cells or normal primary human prostate epithelial cells.

13. The method of any one of claims 1-12, wherein the effective amount is an amount that inhibits osteoclast differentiation.

14. The method of any one of claims 1-13, wherein the MDA-7/IL-24 protein comprises an amino acid sequence that is at least 90% identical to SEQ ID NO: 3.

15. The method of claim 14, wherein the MDA-7/IL-24 protein comprises an amino acid sequence that is at least 95% identical to SEQ ID NO: 3.

16. The method of claim 14 or 15, wherein the MDA-7/IL-24 protein is capable of activating an IL-20/IL-22 receptor complex of a cancer cell of the subject.

17. The method of claim 14, wherein the MDA-7/IL-24 protein comprises an amino acid sequence of SEQ ID NO: 3.

18. A composition comprising an MDA-7/IL-24 protein and one or both of an Mcl-1 inhibitor and a PI3K inhibitor.

19. The composition of claim 18, wherein the MDA-7/IL-24 protein is a purified protein.

20. The composition of claim 18 or 19, wherein the MDA-7/IL-24 protein is a mature protein.

21. The composition of any one of claims 18-20, wherein the composition comprises an Mcl-1 inhibitor.

22. The composition of claim 21, wherein the Mcl-1 inhibitor is BI-97D6.

23. The composition of any one of claims 18-22, wherein the composition comprises a PI3K inhibitor.

24. The composition of claim 23, wherein the PI3K inhibitor is LY294002.

25. The composition of any one of claims 18-24, wherein the MDA-7/IL-24 protein comprises an amino acid sequence that is at least 90% identical to SEQ ID NO: 3.

26. The composition of claim 25, wherein the MDA-7/IL-24 protein comprises an amino acid sequence that is at least 95% identical to SEQ ID NO: 3.

27. The composition of claim 25 or 26, wherein the MDA-7/IL-24 protein is capable of activating an IL-20/IL-22 receptor complex of a cancer cell.

28. The composition of claim 25, wherein the MDA-7/IL-24 protein comprises an amino acid sequence of SEQ ID NO: 3.

29. The composition of any one of claims 18-28, further comprising a pharmaceutically acceptable excipient.

30. The composition of any one of claims 18-29 for use in preventing or treating bone metastasis in a subject with cancer.

31. The composition of claim 30, wherein the cancer is prostate cancer.

32. The composition of claim 31, wherein the prostate cancer comprises cancer cells having an increased expression of one or more of Mcl-1, RANKL, Bcl-2, Bcl-xL, and Akt, relative to normal prostate cells.

33. Use of a composition according to any one of claims 18-29 in the manufacture of a medicament for the prevention or treatment of bone metastasis in a subject with cancer.

34. Use of a composition according to any one of claims 18-29 in the manufacture of a medicament for the prevention or treatment of bone metastasis according to the method of any one of claims 1-17.

35. A kit comprising the MDA-7/IL-24 protein and one or both of the Mcl-1 inhibitor and the PI3K inhibitor of any one of the compositions of claims 18-29.


SEQUENCE LISTING (4 sequences)

SEQ ID NO: 1 - DNA, 621 bp, Artificial Sequence
  Synthetic polynucleotide
atgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggttcctgctattccggagagcatttaaacagttggacgtagaagcagctctgaccaaagcccttggggaagtggacattcttctgacctggatgcagaaattctacaagctctga

SEQ ID NO: 2 - protein, 206 aa, Artificial Sequence
  Synthetic polypeptide
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 3 - protein, 157 aa, Artificial Sequence
  Synthetic polypeptide
QGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 4 - protein, 103 aa, Artificial Sequence
  Synthetic polypeptide
ESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL